(19) United States
(12) Patent Application Publication
(10) Pub. No.: US 2026/0060821 A1
(43) Pub. Date: Mar. 5, 2026

(54) DRUG ELUTING COVERED STENT

(71) Applicant: Boston Scientific Scimed, Inc., Maple Grove, MN (US)

(72) Inventors: Steven L. Kangas, Woodbury, MN (US); Michael Philip Collins, Plymouth, MN (US)

(73) Assignee: Boston Scientific Scimed, Inc., Maple Grove, MN (US)

(21) Appl. No.: 19/317,291

(22) Filed: Sep. 3, 2025

Related U.S. Application Data
(60) Provisional application No. 63/690,597, filed on Sep. 4, 2024.
(60) Provisional application No. 63/853,964, filed on Jul. 30, 2025.

(51) Int. Cl. A61F 2/82

(52) U.S. Cl. CPC A61F 2/82; A61F 2240/001; A61F 2250/0067

(57) ABSTRACT

Covered stents may be coated with or otherwise include a drug eluting composition. An illustrative stent may comprise an elongated tubular body having a strut framework defining a plurality of open cells. A polymeric coating may be disposed on a surface of the elongated tubular body and extending across the plurality of open cells and a first drug coating composition disposed on a surface of the polymeric coating.

### CROSS-REFERENCE TO RELATED APPLICATIONS

This application claims the benefit of priority under 35 U.S.C. § 119 of U.S. Provisional Application No. 63/690,597, filed Sep. 4, 2024, and U.S. Provisional Application No. 63/853,964, filed Jul. 30, 2025, the entire disclosures of which are hereby incorporated by reference.

### TECHNICAL FIELD

The present disclosure pertains to medical devices and more particularly to covered stents including a drug eluting coating composition.

### BACKGROUND

A wide variety of medical devices have been developed for medical use, for example, intravascular and/or intracardiac use. Some of these devices include guidewires, catheters, balloons, stents, and the like. These devices are manufactured by any one of a variety of different manufacturing methods and may be used according to any one of a variety of methods. Some of these medical devices may include a therapeutic agent. Of the known medical devices and methods, each has certain advantages and disadvantages. There is an ongoing need to provide alternative medical devices as well as alternative methods for manufacturing and using medical devices. This may include covered stents that locally deliver a therapeutic agent.

### SUMMARY

The present disclosure pertains to medical devices and more particularly to covered stents including a drug coating.

In an example, a stent may comprise an elongated tubular body having a strut framework defining a plurality of open cells, a polymeric coating disposed on a surface of the elongated tubular body and extending across the plurality of open cells, and a first drug coating composition disposed on a surface of the polymeric coating.

Alternatively, or additionally to any of the examples above, in another example, the first drug coating composition may comprise an excipient and at least one therapeutic agent.

Alternatively, or additionally to any of the examples above, in another example, the at least one therapeutic agent may comprise an anti-proliferative.

Alternatively, or additionally to any of the examples above, in another example, the at least one therapeutic agent may comprise paclitaxel.

Alternatively, or additionally to any of the examples above, in another example, the at least one therapeutic agent may comprise an anti-thrombotic.

Alternatively, or additionally to any of the examples above, in another example, the at least one therapeutic agent may comprise rivaroxaban.

Alternatively, or additionally to any of the examples above, in another example, the excipient may comprise poly(vinylidene fluoride)-co-hexafluoropropylene (PVDF-HFP).

Alternatively, or additionally to any of the examples above, in another example, the polymeric coating may be disposed on a radially outward surface of the elongated tubular body.

Alternatively, or additionally to any of the examples above, in another example, the first drug coating composition may be disposed on a radially outward surface of the polymeric coating.

Alternatively, or additionally to any of the examples above, in another example, the polymeric coating may comprise a first inner layer and a second outer layer.

Alternatively, or additionally to any of the examples above, in another example, the first inner layer and the second outer layer may be formed from a same material.

Alternatively, or additionally to any of the examples above, in another example, the first inner layer and the second outer layer may be formed from different materials.

Alternatively, or additionally to any of the examples above, in another example, the first drug coating composition may be disposed between the first inner layer and the second outer layer.

Alternatively, or additionally to any of the examples above, in another example, the stent may further comprise a second drug coating composition disposed on a radially outward surface of the second outer layer.

Alternatively, or additionally to any of the examples above, in another example, the first drug coating composition may comprise an anti-thrombotic and the second drug coating composition may comprise an anti-proliferative.

In an example, a stent may comprise an elongated tubular body having a strut framework defining a plurality of open cells, a polymeric coating disposed on a surface of the elongated tubular body and extending across the plurality of open cells, and a first drug coating composition disposed on a surface of the polymeric coating.

Alternatively, or additionally to any of the examples above, in another example, the first drug coating composition may comprise an excipient and at least one therapeutic agent.

Alternatively, or additionally to any of the examples above, in another example, the at least one therapeutic agent may comprise an anti-proliferative.

Alternatively, or additionally to any of the examples above, in another example, the at least one therapeutic agent may comprise paclitaxel.

Alternatively, or additionally to any of the examples above, in another example, the at least one therapeutic agent may comprise an anti-thrombotic.

Alternatively, or additionally to any of the examples above, in another example, the at least one therapeutic agent may comprise rivaroxaban.

Alternatively, or additionally to any of the examples above, in another example, the excipient may comprise poly(vinylidene fluoride)-co-hexafluoropropylene (PVDF-HFP).

Alternatively, or additionally to any of the examples above, in another example, the polymeric coating may be disposed on a radially outward surface of the elongated tubular body.

Alternatively, or additionally to any of the examples above, in another example, the first drug coating composition may be disposed on a radially outward surface of the polymeric coating.

Alternatively, or additionally to any of the examples above, in another example, the polymeric coating may comprise a first inner layer and a second outer layer.

Alternatively, or additionally to any of the examples above, in another example, the first inner layer and the second outer layer may be formed from a same material.

Alternatively, or additionally to any of the examples above, in another example, the first inner layer and the second outer layer may be formed from different materials.

Alternatively, or additionally to any of the examples above, in another example, the first drug coating composition may be disposed between the first inner layer and the second outer layer.

Alternatively, or additionally to any of the examples above, in another example, the stent may further comprise a second drug coating composition disposed on a radially outward surface of the second outer layer.

Alternatively, or additionally to any of the examples above, in another example, the first drug coating composition may comprise an anti-thrombotic and the second drug coating composition may comprise an anti-proliferative.

In an example, a stent may comprise an elongated tubular body having a strut framework defining a plurality of open cells, a polymeric coating disposed on a surface of the elongated tubular body and extending across the plurality of open cells, the polymeric coating comprising a first inner layer disposed radially inward of the elongated tubular body and a second outer layer disposed radially outward of the elongated tubular body, and a first drug coating composition disposed between the first inner layer and the second outer layer of the polymeric coating.

Alternatively, or additionally to any of the examples above, in another example, the first inner layer may comprise a small pore size expanded polytetrafluoroethylene (ePTFE).

Alternatively, or additionally to any of the examples above, in another example, the second outer layer may comprise a small pore size expanded polytetrafluoroethylene (ePTFE).

Alternatively, or additionally to any of the examples above, in another example, the stent may further comprise a second drug coating composition disposed on an exterior surface of the second outer layer of the polymeric coating.

In an example, a stent may comprise an elongated tubular body having a strut framework defining a plurality of open cells, a polymeric coating comprising low density expanded polytetrafluoroethylene (ePTFE) disposed on a surface of the elongated tubular body and extending across the plurality of open cells, and a first drug coating composition comprising an excipient and an anti-proliferative disposed on a surface of the polymeric coating.

In an example, a medical device may comprise an elongated tubular body having a strut framework defining a plurality of open cells, a first polymeric film disposed on a luminal surface of the elongated tubular body, wherein the first polymeric film comprises electrospun polytetrafluoroethylene (PTFE), and wherein a first therapeutic agent is infused within pores of the electrospun PTFE.

Alternatively, or additionally to any of the examples above, in another example, the medical device may further comprise a second polymeric film disposed on an abluminal surface of the elongated tubular body, wherein the second polymeric film comprises a second therapeutic agent different from the first therapeutic agent.

Alternatively, or additionally to any of the examples above, in another example, the first polymeric film may comprise a drug-impermeable layer and a drug-containing layer disposed radially inward of the drug-impermeable layer.

Alternatively, or additionally to any of the examples above, in another example, the second polymeric film may comprise a drug-impermeable layer and a drug-containing layer disposed radially outward of the drug-impermeable layer.

Alternatively, or additionally to any of the examples above, in another example, the drug-impermeable layer may comprise styrene-isobutylene-styrene block copolymer (SIBS).

Alternatively, or additionally to any of the examples above, in another example, the first therapeutic agent may comprise an anti-thrombotic agent and the second therapeutic agent may comprise an anti-proliferative agent.

Alternatively, or additionally to any of the examples above, in another example, the anti-thrombotic agent may comprise rivaroxaban and the anti-proliferative agent may comprise paclitaxel.

Alternatively, or additionally to any of the examples above, in another example, the first polymeric film may prevent diffusion of the first therapeutic agent toward an abluminal surface and the second polymeric film may prevent diffusion of the second therapeutic agent toward a luminal surface.

Alternatively, or additionally to any of the examples above, in another example, the second therapeutic agent may be infused within pores of the second polymeric film.

Alternatively, or additionally to any of the examples above, in another example, the medical device may further comprise a polymeric topcoat disposed over at least one of the first polymeric film or second polymeric film.

In an example, a method of manufacturing a drug-eluting medical device may comprise pre-wetting an expanded polytetrafluoroethylene (ePTFE) material with acetone, immediately after pre-wetting, dip coating the ePTFE material in a solution comprising N,N-dimethyl formamide (DMF), a polymer, and a therapeutic agent, removing excess coating from surfaces of the ePTFE material while preserving porosity, and applying the coated ePTFE material to an elongated tubular body having a strut framework.

Alternatively, or additionally to any of the examples above, in another example, the solution may comprise poly(vinylidene fluoride)-co-hexafluoropropylene (PVDF-HFP) and rivaroxaban.

Alternatively, or additionally to any of the examples above, in another example, the method may further comprise applying a topcoat over the coated ePTFE material to control a therapeutic agent release rate.

Alternatively, or additionally to any of the examples above, in another example, removing excess coating may comprise squeegeeing at least one of an inner or outer surface of the ePTFE material.

Alternatively, or additionally to any of the examples above, in another example, the therapeutic agent may equilibrate into pores of the ePTFE material as acetone diffuses out.

In an example, a medical device may comprise an elongated tubular body having a strut framework defining a plurality of open cells, a first polymeric film disposed on a luminal surface of the elongated tubular body, a second polymeric film disposed on an abluminal surface of the elongated tubular body, wherein the first polymeric film comprises a first therapeutic agent configured for luminal release, and wherein the second polymeric film comprises a second therapeutic agent different from the first therapeutic agent configured for abluminal release.

Alternatively, or additionally to any of the examples above, in another example, the first polymeric film may comprise a drug-impermeable layer and a drug-containing layer disposed radially inward of the drug-impermeable layer.

Alternatively, or additionally to any of the examples above, in another example, the second polymeric film may comprise a drug-impermeable layer and a drug-containing layer disposed radially outward of the drug-impermeable layer.

Alternatively, or additionally to any of the examples above, in another example, the drug-impermeable layer may comprise styrene-isobutylene-styrene block copolymer (SIBS).

Alternatively, or additionally to any of the examples above, in another example, the first therapeutic agent may comprise an anti-thrombotic agent and the second therapeutic agent may comprise an anti-proliferative agent.

Alternatively, or additionally to any of the examples above, in another example, the anti-thrombotic agent may comprise rivaroxaban and the anti-proliferative agent may comprise paclitaxel.

Alternatively, or additionally to any of the examples above, in another example, at least one of the first polymeric film or second polymeric film may comprise electrospun polytetrafluoroethylene.

Alternatively, or additionally to any of the examples above, in another example, the first therapeutic agent may be infused within pores of the first polymeric film and the second therapeutic agent may be infused within pores of the second polymeric film.

Alternatively, or additionally to any of the examples above, in another example, the medical device may further comprise a polymeric topcoat disposed over at least one of the first polymeric film or second polymeric film.

In an example, a method of manufacturing a drug-eluting medical device may comprise pre-wetting an expanded polytetrafluoroethylene (ePTFE) material with acetone, immediately after pre-wetting, dip coating the ePTFE material in a solution comprising N,N-dimethyl formamide (DMF), a polymer, and a therapeutic agent, removing excess coating from surfaces of the ePTFE material while preserving porosity, and applying the coated ePTFE material to an elongated tubular body having a strut framework.

Alternatively, or additionally to any of the examples above, in another example, the solution may comprise poly(vinylidene fluoride)-co-hexafluoropropylene (PVDF-HFP) and rivaroxaban.

Alternatively, or additionally to any of the examples above, in another example, the method may further comprise applying a topcoat over the coated ePTFE material to control a therapeutic agent release rate.

Alternatively, or additionally to any of the examples above, in another example, removing excess coating may comprise squeegeeing at least one of an inner or outer surface of the ePTFE material.

Alternatively, or additionally to any of the examples above, in another example, the therapeutic agent may equilibrate into pores of the ePTFE material as acetone diffuses out.

In an example, a stent may comprise an elongated tubular body having a strut framework defining a plurality of open cells, a polymeric covering extending across the plurality of open cells, the polymeric covering comprising an inner layer and an outer layer, a first drug coating composition disposed in a first region of the stent body, a second drug coating composition disposed in a second region of the stent body distinct from the first region, wherein the first and second drug coating compositions comprise different therapeutic agents.

Alternatively, or additionally to any of the examples above, in another example, the first region may comprise a proximal end portion and a distal end portion of the stent body and the second region may comprise a central portion of the stent body.

Alternatively, or additionally to any of the examples above, in another example, the first drug coating composition may be disposed on an abluminal surface and may comprise an anti-proliferative agent.

Alternatively, or additionally to any of the examples above, in another example, the second drug coating composition may be disposed on a luminal surface and may comprise an anti-thrombotic agent.

Alternatively, or additionally to any of the examples above, in another example, the inner and outer layers may comprise ePTFE having different pore sizes to control directional drug release.

Alternatively, or additionally to any of the examples above, in another example, the stent may further comprise an intermediate layer disposed between the inner and outer layers, the intermediate layer comprising fluorinated ethylene propylene (FEP) and disposed radially inward of the strut framework.

Alternatively, or additionally to any of the examples above, in another example, the polymeric covering may extend from a proximal end to a distal end of the stent body to fully cover the tubular body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative endoluminal implant;

FIG. 2 is a partial cross-sectional view of the illustrative implant of FIG. 1;

FIG. 2A is a schematic cross-sectional view of the stent of FIG. 1 having an alternative second layer.

FIG. 2B is a partial cross-sectional view of the illustrative implant of FIG. 1 including an additional layer;

FIG. 3 is a schematic cross-sectional view of the stent of FIG. 1 having an alternative coating configuration;

FIG. 4 is a graphical representation of experimental data providing relative drug release rates for different laminates using various grades of ePTFE having differing pore sizes;

FIG. 5 is a graphical representation of experimental data providing relative drug release rates for samples made with electrospun PTFE;

FIG. 6 is a graphical representation of experimental data providing relative drug release rates for samples made at least in part with a PET woven mesh;

FIG. 7 is a graphical representation of experimental data providing relative drug release rates for samples made at least in part with microporous PVDF;

FIG. 8 is a graphical representation of experimental data providing relative drug release rates for samples made at least in part with ePTFE and a layer of PVDF-HFP;

FIG. 9 is a graphical representation of experimental data providing relative drug release rates for samples made with ePTFE at varying lamination pressures;

FIG. 10 is a graphical representation of in-vitro drug release rates (in nanograms (ng)/mm²/day) for an ePTFE/PVDF-HFP-rivaroxaban/ePTFE construction;

FIG. 11 is a graphical representation of experimental data providing relative drug release rates for a covered stent that is abluminally coated (e.g., radially outwardly coated) with a PVDF-HFP and paclitaxel drug coating composition;

FIG. 12 is a graphical representation of experimental data providing in-vitro drug release rate (in percent of drug released) for an ePTFE covered stent (including both abluminal and luminal ePTFE coverings);

FIG. 13 is a graphical representation of experimental data providing in-vitro drug release rate for the covered stent construction of the data of FIG. 12;

FIGS. 14A-14E depict cross-sectional schematic views of the process steps for dip coating an ePTFE fabric to deposit a drug coating in the pores thereof;

FIG. 15 is a graphical representation of experimental data providing average drug release percentages for covered stents including an abluminal paclitaxel coating;

FIG. 16 is a graphical representation of experimental data providing average drug release percentages for the fully covered stents including an abluminal and luminal paclitaxel coating, the partially covered stents with drug coated ends, and an Eluvia™ control;

FIG. 17 is a graphical representation of in vitro drug release of the covered Eluvia™ compared to the uncovered Eluvia™ control;

FIG. 18 is a graphical representation of experimental data providing relative drug release rates for a dual eluting stent; and

FIG. 19 is a partial cross-sectional schematic of a dual eluting covered stent.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following detailed description should be read with reference to the drawings in which similar elements in different drawings are numbered the same. The drawings, which are not necessarily to scale, depict illustrative embodiments and are not intended to limit the scope of the invention.

As used herein, the term “disposed on” or “disposed in” includes configurations where a drug coating composition is positioned directly on a surface of a polymeric coating, as well as configurations where the drug coating composition equilibrates into or penetrates the polymeric coating. The drug coating composition may be applied to an exterior and/or interior surface and subsequently migrate, diffuse, or equilibrate into pores, interstices, or the bulk material of the polymeric coating while maintaining therapeutic efficacy.

Expanded polytetrafluoroethylene (ePTFE) covered stents may be used in the treatment of peripheral artery disease (PAD). For example, covered stents may have special utility in cases where there is a long diseased artery or there is a dissection or an aneurysm is present. Although stent patency of covered stents may be better than stent patency of uncovered stents, stent thrombosis and restenosis do occur in covered stents. In the case of covered stents used to treat an arteriovenous (AV) fistula or a graft failure, the rate of covered stent thrombosis and/or restenosis may be significant. It may be desirable to provide a covered stent that reduces the risk of thrombus formation and restenosis.

While the therapeutic coating, covering, or layer described herein is discussed relative to covered stents, it is contemplated that the therapeutic coating, covering, or layer can be applied to and/or used in conjunction with other medical devices, such as, but not limited to, bare metal stents, uncovered stents, balloons, balloon catheters, embolic filters, implantable devices, treatment devices, etc.

The medical devices disclosed herein may provide anti-proliferatives and/or factor Xa (FXa) inhibitors (such as, but not limited to, direct oral anticoagulants (DOACs)) on the surface of a device. The device may then be delivered to where the local treatment is needed. The FXa inhibitor may reduce the risk of thrombus formation while the anti-proliferative may reduce the risk of neointimal hyperplasia (NIH), restenosis, scar formation, or the like. Coated stents having an anti-proliferative and/or a factor Xa inhibitor may be used to treat PAD in the superficial femoral artery (SFA), the iliacs, and/or the aortoiliacs, arteriovenous fistulas, graft failures, or the like.

The terms “therapeutic agents,” “drugs,” “bioactive agents,” “pharmaceuticals,” “pharmaceutically active agents”, and other related terms may be used interchangeably herein and include genetic therapeutic agents, non-genetic therapeutic agents, and cells. Therapeutic agents may be used singly or in combination. A wide range of therapeutic agent loadings can be used in conjunction with the devices of the present disclosure, with the pharmaceutically effective amount being readily determined by those of ordinary skill in the art and ultimately depending, for example, upon the condition to be treated, the nature of the therapeutic agent itself, the tissue into which the dosage form is introduced, and so forth.

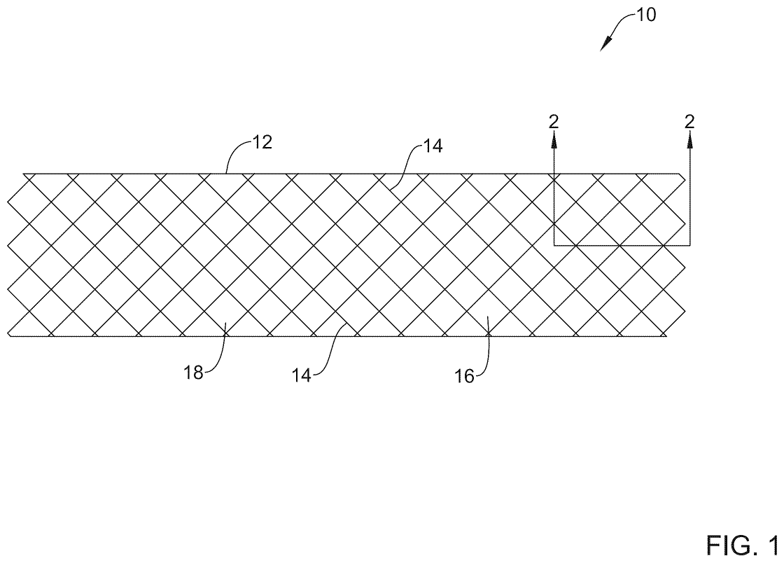

FIG. 1 is a schematic side view of an illustrative drug-eluting endoluminal implant, such as, but not limited to a drug-eluting stent 10 in an expanded or deployed configuration. In general, the stent 10 may be delivered to a suitable target region via a catheter/delivery system while in a radially collapsed configuration (not explicitly shown). Upon reaching the target region, the stent 10 may expand or be expanded into the expanded configuration. The stent 10 may be self-expanding (e.g., the stent 10 may be formed from a shape memory material such as nitinol) or may be balloon expandable. When the stent 10 is self-expanding, the stent 10 may be held/constrained in the collapsed configuration during delivery and then unconstrained to allow the stent 10 to expand (e.g., self-expand) to the expanded configuration. When the stent 10 is balloon-expandable, the stent 10 may be crimped onto a delivery device/catheter and then expanded (e.g., via an expandable member or balloon) when at/adjacent to the target region.

The stent 10 may include an elongated tubular body 12. The stent 10 may have a woven structure, fabricated from a number of filaments or struts 14 forming a tubular wall. In some embodiments, the stent 10 may be knitted or braided with a single filament or strut interwoven with itself and defining open cells 16 extending through the thickness of the tubular wall of the stent 10. In other embodiments, the stent 10 may be braided with several filaments or struts interwoven together and defining open cells 16 extending along a length and around the circumference of the tubular wall of the stent 10. In yet another embodiment, the stent 10 may be of a knotted type. In still another embodiment, the stent 10 may be a laser cut tubular member. A laser cut tubular member may have an open and/or closed cell geometry including one or more interconnected monolithic filaments or struts defining open cells 16 therebetween. The open cells 16 may each define an opening from an outer surface of the tubular wall to an inner surface of the tubular wall (e.g., through a thickness thereof) that is free from the filaments or struts 14.

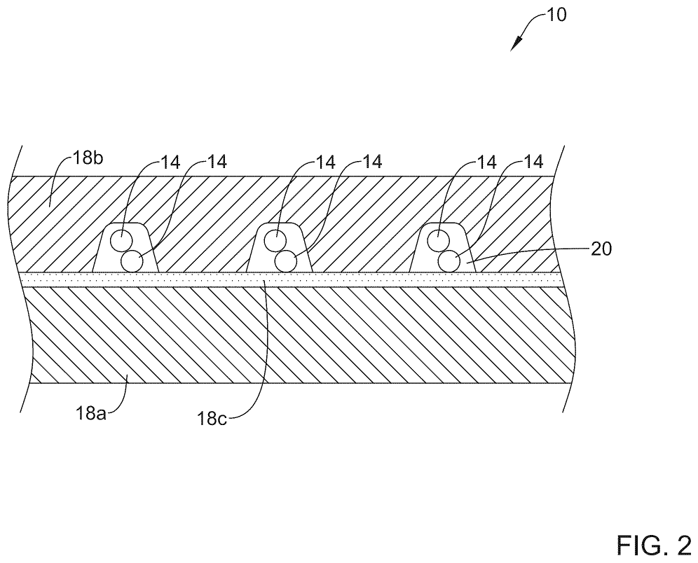

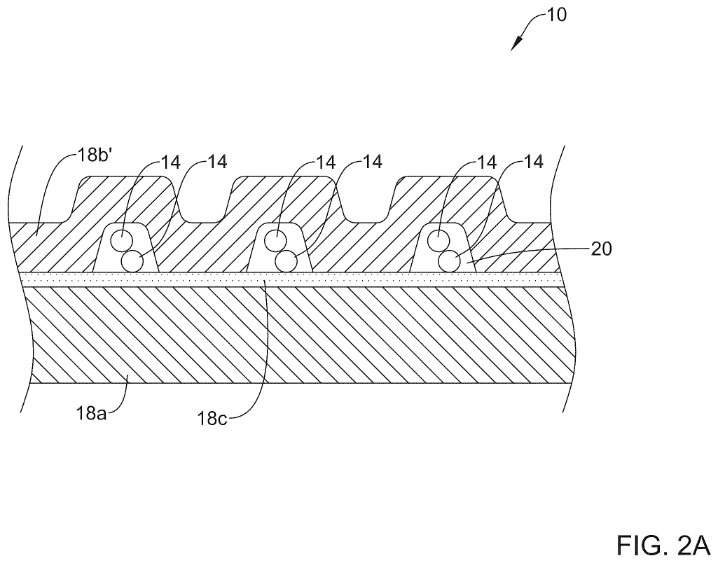

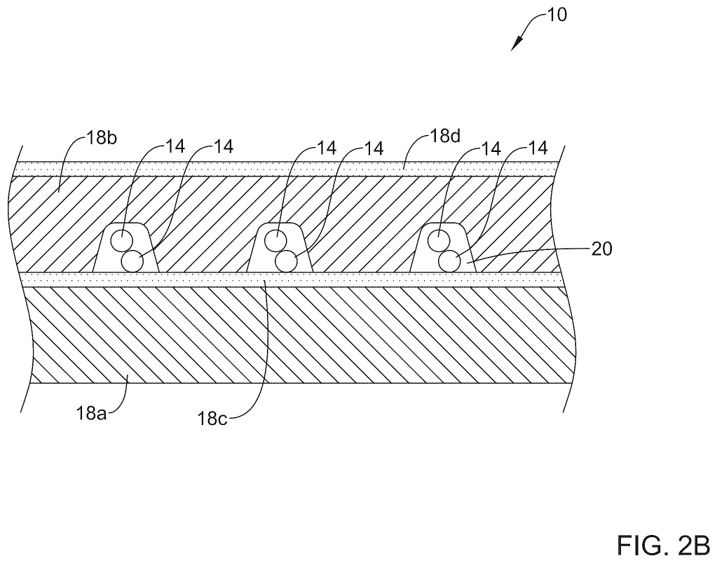

In some instances, an inner and/or outer surface of the tubular wall of the stent 10 may be entirely, substantially, or partially, covered with a polymeric covering or coating 18. As will be described in more detail herein, the covering or coating 18 may include one or more layers of polymers and/or drug compositions. The covering or coating 18 may extend across and/or occlude one or more, or a plurality of the cells 16 defined by the struts or filaments 14. FIG. 2 is a partial schematic cross-sectional view of the illustrative stent 10, taken at line 2-2 of FIG. 1. For example, the covering or coating 18 may extend along an entire length or along only a portion of the length of the stent 10. Similarly, the covering or coating 18 may extend around an entirety of the circumference of the stent 10 or around only a portion of the circumference of the stent 10. Further, the covering or coating 18 need not be continuous along a length and/or around a circumference of the stent 10. For example, two or more regions including a covering or coating 18 may be axially and/or circumferentially separated by one or more regions free from the covering or coating 18.

In some embodiments, the stent 10 may include a first or inner layer 18a, a second or outer layer 18b, and a third or tie layer 18c positioned between the first layer 18a and the second layer 18b. It is contemplated that one or more of the first layer 18a, the second layer 18b, or the third layer 18c may be omitted and/or repositioned. The first and second layers 18a, 18b may be formed from the same material or different materials, as desired. In some embodiments, the first and/or second layers 18a, 18b may be formed from ePTFE. Other illustrative materials for the first and/or second layers 18a, 18b may include, but are not limited to polyethylene terephthalate (PET), a woven mesh PET, poly(vinylidene fluoride) (PVDF), microporous PVDF, poly(vinylidene fluoride)-co-hexafluoropropylene (PVDF-HFP), or the like. It is contemplated that the third layer 18c may be formed from a material different from the first and second layers 18a, 18b to help facilitate adhesion. However, this is not required. In some embodiments, the third layer 18c may not function as a tie layer or to facilitate adhesion. In some embodiments, the third layer 18c may be formed from a thermoplastic tie layer such as, but not limited to, fluorinated ethylene propylene (FEP), ePTFE, PVDF, PVDF-HFP. The third layer 18c may be formed from a same material as one of the first or second layers 18a, 18b, as desired. In one illustrative example, the first and second layers 18a, 18b may be melt bonded ePTFE with a third layer 18c of FEP therebetween. The filaments or struts 14 may be positioned between the first and second layers 18a, 18b. The first layer 18a, the second layer 18b and/or the third layer 18c may span or be disposed within openings or interstices defined between adjacent stent filaments or struts 14 of the scaffold structure. The covering or coating 18 may extend between adjacent filaments or struts 14 and thus prevent tissue ingrowth into the lumen of the stent 10.

In the illustrated embodiment, the third layer 18c may extend along the luminal surface of the stent body 12. For example, the third layer 18c may be disposed radially inwards of the filaments or struts 14. The first layer 18a may be adhered to, or otherwise coupled with, the radially inward surface of the third layer 18c. The second layer 18b may be adhered to, or otherwise coupled with, the radially outward surface of the third layer 18c. In some examples, the second layer 18b may be formed such that the stent 10 has a uniform outer diameter. In this example, the second layer 18b may fully or partially encapsulate the filaments or struts 14. Said differently, the second layer 18b may fill in any gaps between overlapping filaments or struts 14 and may contact an outer surface of the filaments or struts 14. In some cases, the second layer 18b may not contact an entirety of the filaments or struts 14. For example, there may be gap or open space 20 between portions of the filaments or struts 14 and the inner surface of the second layer 18b. The thickness of the second layer 18b may vary along a length and/or around circumference of the stent 10. For example, the thickness of the second layer 18b adjacent to a filament or strut 14 may be less than a thickness of the outer layer adjacent to an open cell 16.

In other embodiments, the thickness of the second layer 18b may be uniform along a length and/or around a circumference of the stent 10. FIG. 2A illustrates a schematic cross-sectional view of the stent 10 having an alternative second layer 18b′. In FIG. 2A, the second layer 18b′ has a uniform thickness as it is applied to the outer surface of the third layer 18c and the stent body 12. The second layer 18b′ may conform to the profile of outer surface of the stent 10 such that the second layer 18b′ undulates or rises and falls along a length and/or around a circumference of the stent 10. In some examples, there may be gap or open space 20 between portions of the filaments or struts 14 and the inner surface of the second layer 18b′. However, this is not required. In some examples, the second layer 18b′ may encapsulate, surround, fill in gaps, etc., if so desired. It should be understood that any of the layers (e.g., first, second, third, and the like) described herein may have a varying thickness (FIG. 2) or a uniform thickness (FIG. 2A), or combinations thereof along the length and/or around the circumference of the stent 10.

A person of ordinary skill in the art could appreciate that a vast number of alternate configurations of the layers 18a, 18b, 18c may be included without departing from the scope of the disclosure. Some illustrative covered stents are described in commonly assigned U.S. Patent Publication Number 2024/014847, titled STENT WITH ANTI-MIGRATION FEATURES and U.S. Patent Ser. No. 63/514,024, titled STENT WITH ANTI-OCCLUSION SYSTEM, the disclosures of which are hereby incorporated by reference.

Returning to FIG. 2, one or more of the layers 18a, 18b, 18c may include one or more therapeutic agents incorporated therein. For example, one or more of the layers 18a, 18b, 18c may be a drug composition such as, but not limited to, a drug coating composition or a drug eluting composition, including at least a therapeutic agent and an excipient. It is contemplated that the therapeutic agent may be incorporated into a polymer matrix. Positioning, coating or otherwise disposing a therapeutic agent directly on the radially outward surface of the second layer 18b or the radially inward surface of the first layer 18a may reduce or eliminate the advantages of the microstructure of ePTFE. However, in applications where obstructing the microstructure of the ePTFE will not adversely impact the function of the stent 10, a therapeutic coating or therapeutic agent may be applied directly to the inner (luminal) and/or outer (abluminal) surface of the stent 10. The first layer 18a, second layer 18b, and/or third layer 18c may include a therapeutic agent that includes an anti-thrombotic, such as, but not limited to, apixaban, rivaroxaban, edoxaban, dabigatran, betrixaban, and argatroban or an anti-proliferative, such as, but not limited to, paclitaxel. Additionally, or alternatively, the first layer 18a, second layer 18b, and/or third layer 18c may include other beneficial therapeutic agents such as, but not limited to, anti-thrombotic agents, other anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others, and/or combinations thereof.

In some embodiments, the drug may be a macrolide immunosuppressive (limus) drug. In some embodiments, the macrolide immunosuppressive drug is rapamycin, biolimus (biolimus A9), 40-O-(2-Hydroxyethyl) rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4 (S)-yl)-prop-2′-cn-1′-yl]-rapamycin, (2′: E,4'S)-40-O-(4′,5′-Dihydroxypent-2′-en-1′-yl)-rapamycin, 40-O-(2-Hydroxy) ethoxycar-bonylmethyl-rapamycin, 40-O-(3-Hydroxy) propyl-rapamycin, 40-O-(6-Hydroxy) hexyl-rapamycin, 40-O-[2-(2-Hydroxy) ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino) acetoxy]ethyl-rapamycin, 40-O-(2-N-Imidazolylacetoxy)ethyl-rapamycin, 40-O-[2-(N-Methyl-N′-piperazinyl) acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminocthyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidocthyl)-rapamycin, 40-O-[2-(4′,5′-Dicarbocthoxy-1′,2′,3′-triazol-1′-yl)-ethyl]-rapamycin, 42-Epi-(tetrazolyl) rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), or derivative, isomer, racemate, diastereoisomer, prodrug, hydrate, ester, or analog thereof. Other drugs may include anti-inflammatory agents such as dexamethasone, prednisolone, corticosterone, budesonide, estrogen, sulfasalazine, mesalamine, and analogues thereof; antineoplastic/anti-proliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

In an embodiment, the third layer 18c may be a drug eluting composition including an excipient and a therapeutic agent. Excipients can be used to enhance the durability of the drug composition, facilitate drug transfer to the lesion or desired treatment location, and/or control drug dissolution. An example excipient may include poly(vinylidene fluoride)-co-hexafluoropropylene (PVDF-HFP) which can be formed as a porous membrane. Other example excipients may include, but are not limited to, acetyl tri-butyl citrate (ATBC), polylactic acid (PLA), acetyl tri-butyl citrate (ATHC), poly-DL-lactide (PDLLA), poly-L-lactide (PLLA), poly-D-lactide (PDLA), poly(lactic-co-glycolic acid) (PLGA), etc. It is contemplated that the release rate of rivaroxaban, paclitaxel, and/or other therapeutic agent can be increased or decreased based on the degradation rate of the excipient. For example, PLGA may degrade faster than PLA. In some examples, additional components may also be used in the drug coating composition. In some cases, an antioxidant, such as, but not limited to, butylated hydroxytoluene (BHT) may be used in the composition.

In some instances, the drug coating composition may include individual drug particles that are encapsulated with one or more excipients. The drug particles may include crystals of the drug, for example. Drug crystals may be formed in a variety of ways, for example. In some cases, a drug or other therapeutic agent may be available in an amorphous form, and a variety of processes may be used to convert an amorphous drug or other therapeutic agent into a crystalline drug or other therapeutic agent. In other examples, the therapeutic agent may be available in crystalline form. In yet other examples, the therapeutic agent may become crystalline during the process of making and applying the drug coating composition. However, in some cases, the individual drug particles need not be encapsulated.

In one illustrative embodiment, the drug composition of the third layer 18c may include a DOAC, such as, but not limited to, rivaroxaban, as the therapeutic agent with PVDF-HFP as the excipient. In another example, the drug composition of the third layer 18c may include an anti-proliferative, such as but not limited to, paclitaxel, as the therapeutic agent with PVDF-HFP as the excipient. In some examples, the PVDF-HFP may have a VDF to HFP ratio of 85/15. However, this is not required. Other ratios may be used as desired. The therapeutic agent may form crystalline particles within a continuous PVDF-HFP matrix. The resulting drug composition 18c may be disposed between the first layer 18a and the second layer 18b to act as a drug depot for sustained localized release. When the drug composition is disposed between the first and second layers 18a, 18b (e.g., the drug composition is a third layer 18c, intermediate layer, etc.), the release of the therapeutic agent may be modulated by the first and/or second layers 18a, 18b. For example, the therapeutic agent may be released luminally (e.g., within or towards the lumen of the stent 10 to the blood contact surface of the first layer 18a) and/or abluminally (e.g., towards the body tissue of the vessel or lumen wall of the implant location) depending on the properties of the first and/or second layers 18a, 18b.

In one embodiment, a polymer coating such as PVDF-HFP, and one or more anti-thrombotics and/or one or more anti-proliferatives are dissolved in a solvent suitable for dissolving the polymer and drug. In some examples, the solvent may be a blend of acetone and N,N-dimethyl formamide (DMF). However, other solvents such as, but not limited to, N,N-dimethyl acetamide (DMAc), dimethylsulfoxide (DMSO) and N-methyl pyrrolidone (NMP), may be used. This solution may be applied directly to the stent 10 by a dip coating or spray process. It is contemplated that an entirety of the stent 10 may include the drug composition 18c. In other examples, less than an entirety of the stent 10 may include the drug composition 18c. The drug composition 18c may be applied to achieve a coating density of between 10-4,000 nanograms (ng) drug per square millimeter (mm²) (ng/mm²) on the surface of the stent 10 (based on vessel surface area). The drug composition 18c may include in the range of about 5 weight percent to about 45 weight percent therapeutic agent and in the range of about 55 weight percent to about 95 weight percent PVDF-HFP (or other excipient). The drug composition 18c may have a thickness of up to 10 micrometers (μm) and therapeutic agent content of in the range of about 100-300 micrograms (μg) may be achievable. In another example, the amount of drug contained in the drug composition 18c may be about 10-4000 ng/mm². The amount of drug contained in the drug composition 18c may vary depending on the type of drug within the drug composition 18c. It is contemplated that a drug composition 18c including PVDF-HFP and a DOAC, such as rivaroxaban, may release in the range of about 40-80% of the therapeutic agent in about 7-60 days when the device 10 is implanted.

As described above, in some cases, the first and/or second layers 18a, 18b may be formed from ePTFE (a porous material). In order to allow for drug release from the third layer 18c, the first and/or second layers 18a, 18b should allow for diffusion of blood, the aqueous components of blood (e.g., plasma or water), or other bodily fluid to the third layer 18c or drug composition. A feature of ePTFE is that ePTFE is widely known to be hydrophobic (e.g., at a water contact angle of) 140° and impermeable to water. Unexpectedly, the inventor of the present disclosure has found that while ePTFE modulates the release of the therapeutic agent from the third layer 18c, clinically relevant amounts of therapeutic agent may be released through an ePTFE first layer 18a and/or an ePTFE second layer 18b. Luminal only release may be accomplished by utilizing a second layer 18b of ePTFE of small pore size to prevent and/or minimize drug release. Abluminal only release may be accomplished by utilizing a first layer 18a of ePTFE of very low pore size to prevent and/or minimize drug release. Said differently, the stent 10 may include at least one layer 18a, 18b which allows for diffusion of the therapeutic agent therethrough and at least one layer 18a, 18b which prevents or substantially reduces diffusing of the therapeutic agent therethrough. In some embodiments, ePTFE having an internodal distance of less than 50 μm may be effective at preventing or reducing diffusion of the therapeutic agent. It is contemplated that by placing or positioning the drug composition between the first and second layers 18a, 18b, the morphology of the ePTFE layer may not be affected.

In one illustrative example, both the first layer 18a and the second layer 18b may be formed from ePTFE. The morphology of the first layer 18a may be different from the morphology of the second layer 18b. For example, when the third layer 18c includes an anti-proliferative, such as, but not limited to, paclitaxel as the therapeutic agent, it may be desired for the therapeutic agent to be released radially outwards towards or into the vessel wall of the implant location (abluminally) to reduce the risk of neointimal hyperplasia. In such an instance, the second (outer) layer 18b of ePTFE may be more porous than the first (inner) layer 18a of cPTFE. Alternatively, when the third layer 18c is an anti-thrombotic, such as, but not limited to, rivaroxaban, it may be desired for the therapeutic agent to be released radially inwards towards the lumen of the stent 10 (luminally) to reduce the risk of thrombus formation. In such an instance, the first (inner) layer 18a of ePTFE may be more porous than the second (outer) layer 18b of cPTFE.

It is contemplated that the layer through which the therapeutic agent is desired to diffuse, the therapeutic agent modulating layer, may be formed from materials other than ePTFE. In one example, the first layer 18a and/or second layer 18b may be formed from a PET woven mesh. It is contemplated that a PET woven mesh (or other PET structure) may be coated with a thin layer of PVDF to increase biocompatibility. However, this is not required. In another example, the first layer 18a and/or second layer 18b may be formed from a microporous PVDF. It is contemplated that directional release of two different therapeutic agents may be achieved by separating two differing drug composition with a layer of ePTFE having small pore size and covering each drug composition with a layer of ePTFE (or other material, such as, but not limited to, PET woven mesh or microporous PVDF). Such a stent 10 may have five layers. For example, an outermost layer may include a high porosity (or large pore size) ePTFE. The next radially inward layer may be a drug composition including an anti-proliferative. Following this layer, the next radially inwards layer may be a lower porosity (or small pore size) ePTFE. The next layer may be a drug composition including an anti-thrombotic. The radially inward most layer may include a high porosity ePTFE. This may allow the stent 10 to directionally elute or release the therapeutic agents to or towards the desired anatomical location(s).

Referring additionally to FIG. 2B, which is the cross-sectional view of FIG. 2 including an additional optional layer 18d, in some examples, the stent 10 may include two or more drug compositions. For example, the third layer 18c may include a first drug composition and the optional layer 18d may include a second drug composition disposed over the second layer 18b. In the embodiment of FIG. 2B, the second drug composition layer 18d may be the radially outward most portion of the stent 10 on a radially outwards surface of the second layer 18b while the first drug composition layer 18c may be disposed between the first (radially inward most) layer 18a and the second layer 18b. It is contemplated that the second drug composition layer 18d may be different from the first drug composition layer 18c. For example, the second drug composition layer 18d may include an anti-proliferative, such as, but not limited to, paclitaxel, as the therapeutic agent. When the stent 10 is deployed, the second drug composition layer 18d may be positioned in apposition with the vessel wall such that the therapeutic agent (e.g., anti-proliferative) may be released radially outwards into the tissue of the vessel wall at the implant location (abluminally) to reduce the risk of neointimal hyperplasia. The first drug composition layer 18c may include an anti-thrombotic, such as, but not limited to, rivaroxaban, as the therapeutic agent. The second layer 18b may be formed from a small pore size ePTFE while the first layer 18a may be formed from a larger pore size ePTFE, an electrospun PTFE, a PET woven mesh, a microporous PVDF, or other polymer which allows for the diffusion of blood through the first layer 18a towards the first drug composition layer 18c. Thus, the therapeutic agent (anti-thrombotic) to be released radially inwards towards the lumen of the stent 10 (luminally) to reduce the risk of thrombus formation.

In some embodiments, the second drug composition layer 18d may include only the therapeutic agent. For example, a therapeutic agent, such as, but not limited to, paclitaxel, may be applied directly to the outer surface of the second layer 18b. Said differently, the second drug composition layer 18d may be free from excipients, anti-oxidants, or other additives. In some embodiments, the second drug composition layer 18d may not extend along an entire length and/or circumference of the stent 10. In one illustrative example, the second drug composition layer 18d may include a first portion positioned adjacent to a proximal end of the stent 10 and a second portion positioned adjacent to a distal end of the stent 10. The region between the first and second portions may be free from the second drug composition 18d. Such a configuration may allow for targeted release of the anti-proliferative directly where stenosis is likely to occur.

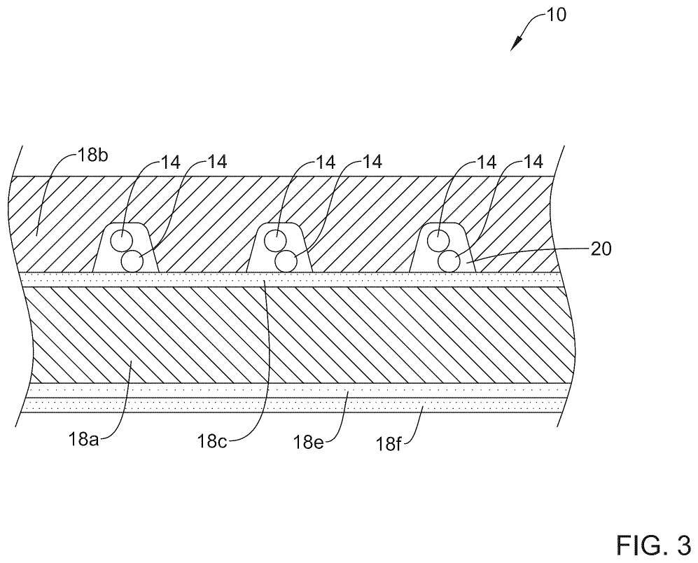

FIG. 3 is the cross-sectional view of FIG. 2 including two additional optional layers 18c, 18f. In some examples, the stent 10 may include two or more drug compositions. For example, the third layer 18c may include a first drug composition and the optional layer 18e may include a second drug composition. A drug coating composition layer 18e may extend along a radially inward surface of the first layer 18a. The drug coating composition layer 18e may be any of the drug coating compositions described herein. In one illustrative example, the drug coating composition layer 18e may include an anti-thrombotic with PVDF-HFP as the excipient. However, this is not required. The drug coating composition layer 18e may include other therapeutic agents and/or excipients, as desired. A modulating layer 18f may extend along a radially inward surface of the drug coating composition 18c. The modulating layer 18f may be a layer of polymer, such as, but not limited to PVDF-HFP, that is deposited over the drug coating composition layer 18e to modulate the release rate of the therapeutic agent. It is contemplated that increasing the weight percent of the polymer topcoat relative to the weight of the drug coating composition layer 18e may slow a release of the therapeutic agent. Alternatively, or additionally, a drug coating composition layer may extend along a radially outward surface of the second layer 18b with a modulating polymeric layer disposed over the radially outward surface of said drug coating composition layer. When a drug coating composition layer is disposed over the second layer 18b, the therapeutic agent may include an anti-proliferative.

It is contemplated that the duration and/or quantity of drug release may be modulated to achieve the desired result. In some cases, the duration of the drug release may be controlled from days to weeks by controlling formulation parameters. For example, the polymer excipient to therapeutic agent ratio may be increased or decreased to achieve a desired release profile. Further, the porosity (pore size) and/or thickness of a polymer or modulating layer 18a, 18b, 18f over the drug coating composition 18c, 18d, 18e may be varied to adjust the drug release profile. For example, increasing a porosity or pore size of the polymer layer 18a, 18b, 18f may increase diffusion and thus increase drug release. In contrast, decreasing a porosity or pore size of the polymer layer 18a, 18b, 18f may decrease diffusion and thus reduce or prevent drug release. It is further contemplated that as the thickness of the polymer layers 18a, 18b, 18f increases, the drug release rate may decrease. The presence of a polymer or modulating layer 18a, 18b, 18f may also be used to vary the drug release profile from the drug coating composition 18c, 18d, 18c. For example, a drug coating composition 18c, 18d, 18e having a polymer or modulating layer 18a, 18b, 18f disposed thereover may release the therapeutic agent slower than when no polymer or modulating layer 18a, 18b, 18f is present. In yet another example, lamination pressure during application of the layers 18a-f may be used to modulate the drug release. For example, a higher lamination pressure may cause the drug coating composition 18c between layers 18a, 18b to penetrate deeper into the first and/or second layers 18a, 18b. This may increase the drug layer surface area and create a shorter path for drug diffusion which may result in an increase in drug release rates.

### Process for Forming Crystalline Rivaroxaban

Crystalline rivaroxaban may be generated by crystallization of the rivaroxaban from a solution of a mixture of N,N-dimethyl formamide (DMF) and water. Crystalline rivaroxaban may also be generated by slowly adding a solution of rivaroxaban in DMF (1 to 4 wt % solids) into a large excess of deionized (DI) water.

### EXPERIMENTAL RESULTS

In one illustrative example, a drug eluting covered stent 10 including rivaroxaban was manufactured with a laminate coating. For example, a ten-weight percent (wt %) solids solution of PVDF-HFP and rivaroxaban (96/4 weight/weight (wt/wt)) in 60/40 acetone and dimethylformamide (DMF) (wt/wt) solvent was coated on glass using a knife coater. The coating was dried at 125° C. for 15 minutes in a convection oven. The resulting coating (approximately 14 micrometers (μm) thick) was peeled off from the glass plate and the film was placed in contact with an ePTFE film having a thickness of approximately 30-40 μm and an internodal distance of approximately 20 μm internodal distance. The polymer-drug coating composition and the ePTFE films were placed between glass plates, clamped together using a clamp force of approximately 1400 grams (g) and placed in an oven at 200° C. for five minutes. The resulting polymer-drug coating composition and ePTFE laminate was wrapped around an approximately 7.6 millimeter (mm) diameter mandrel with the polymer-drug coating composition layer on the outside and the ePTFE layer in contact with the mandrel. An 8.0 mm diameter by 60 mm long nitinol stent was then slid over the laminate wrap. An ePTFE film having a thickness of approximately 30-40 μm and an internodal distance of approximately 20 μm was then wrapped around the outside of the stent. The entire construction was tightly wrapped with PTFE tape to ensure good interlayer contact between the laminate film (e.g., polymer-drug coating composition and ePTFE laminate) within the inner diameter of the stent and the ePTFE layer surrounding the outer diameter of the stent. The construction was placed in a convection oven at 200° C. for five minutes. After cooling, the PTFE tape wrap was removed and the stent was removed from the mandrel. A covered stent including a drug eluting layer between layers of ePTFE resulted. It is contemplated that two or more polymer-drug coating compositions may be used to provide more than one type of therapeutic agent within a single layer. For example, when the polymer/drug/solvent mixture is coated on the glass, a first region of the glass may include a first type of therapeutic agent and a second region of the glass may include a second type of therapeutic agent. Said differently, more than one polymer/drug/solvent mixture may be applied to the glass. In one illustrative example, one mixture may include an anti-proliferative, such as, but not limited to paclitaxel, and another mixture may include an anti-thrombotic, such as, but not limited to, rivaroxaban. The two or more mixtures may be applied to the glass such that the mixture including the anti-proliferative is positioned on either side of the anti-thrombotic. When the resulting laminate is wrapped around the mandrel, the anti-proliferative may be disposed at or adjacent to the proximal and/or distal ends of the stent while the anti-thrombotic is disposed therebetween.

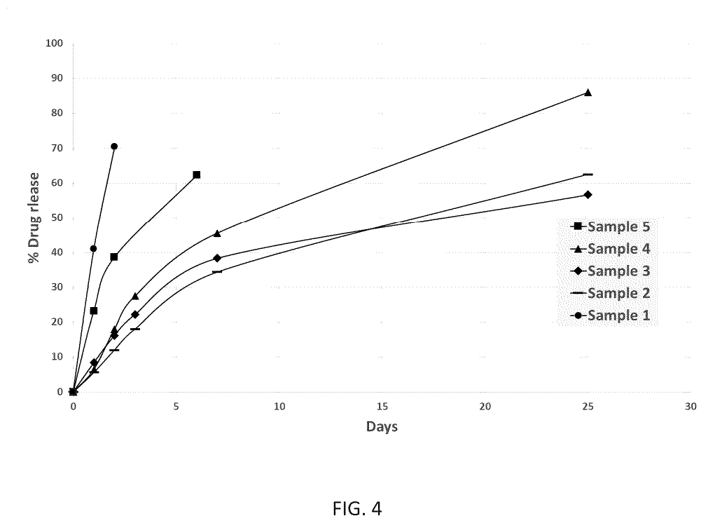

FIG. 4 is a graphical representation of experimental data providing relative drug release rates for different laminates using various grades of ePTFE having differing porosities (e.g., differing internodal distances). Films of PVDF-HFP-rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 micrometers (μm) thick) was peeled off from the glass plate. The resulting PVDF-rivaroxaban films were laminated between two layers of ePTFE using a clamp force of approximately 1400 g at 200° C. for five minutes. The same ePTFE was used for each layer. Said differently, five different samples were prepared with the porosity of the ePTFE varying across samples. However, the porosity of each layer of ePTFE used in a same sample was held constant. Expanded PTFEs of different porosities were generated by stretching the PTFE ribbon at various stretch ratios at 320° C. and a high stretch rate of about 3 feet per second (ft/sec). For example, increasing the stretch length increases the internodal distance of the ePTFE. Five different laminate samples were prepared with varying ePTFE porosities. Table 1 shows the internodal distance in μm of the ePTFE in each sample. The control did not include ePTFE layers.

TABLE 1
Porosity of ePTFE for each laminate

| | ePTFE internodal distance |
|---|---|
| Sample | (um) |
| 1 | Control (no ePTFE) |
| 2 | 17 |
| 3 | 56 |
| 4 | 75 |
| 5 | 100-150 |

Disks were cut from the laminate constructions and drug release was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 2 shows the percent of drug release over time.

TABLE 2
Percent of drug released over time

| | Number of Days | | | | | |
|---|---|---|---|---|---|---|
| | 1 | 2 | 3 | 6 | 7 | 25 |
| Sample 1 | 41% | 70.4% | 85.4% | — | 96.5% | — |
| Sample 2 | 5.6% | 12% | 18% | — | 34.4% | 62.5% |
| Sample 3 | 8.4% | 16.2% | 22.2% | — | 38.3% | 56.7% |
| Sample 4 | 6.5% | 17.9% | 27.5% | — | 45.5% | 86% |
| Sample 5 | 23.1% | 38.6% | — | 62.3% | — | — |

As can be seen in Table 2 and FIG. 4, the ePTFE modulated the release of the drug. For example, sample 1 which did not include ePTFE layers on either side of the polymer-drug composition released the drug the quickest. Further, as the internodal distance increased (and thus increasing the porosity and reducing the density of the ePTFE), the rate of drug released also increased. For example, Sample 5 with the largest internodal distance released the drug the quickest of the samples which included an ePTFE layer but not faster than Sample 1 (control) which did not include an ePTFE layer while Sample 2 with the smallest internodal distance released the drug the slowest.

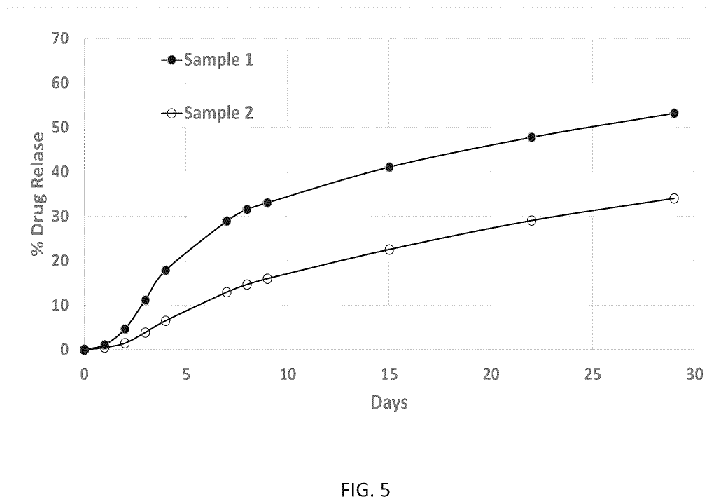

FIG. 5 is a graphical representation of experimental data providing relative drug release rates for samples made with electrospun PTFE and incubated in PBS (phosphate buffered saline) with 0.05% TWEEN® 20, pH 7.4 media (available from Sigma-Aldrich based in St. Louis, MO) at 37° C. Films of PVDF-HFP/rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 micrometers (μm) thick) was peeled off from the glass plate. The resulting PVDF-rivaroxaban films were laminated between two layers of PTFE using a clamp force of approximately 1400 g at 200° C. for five minutes. In Sample 1, the polymer-drug composition was laminated between two layers of electrospun PTFE. In Sample 1, the therapeutic agent may release from both sides of the sample (two side release). In Sample 2, the polymer-drug composition was laminated between a layer of electrospun PTFE and a layer of small pore size ePTFE. In Sample 2, the therapeutic agent may primarily release through the electrospun PTFE (one side release). Table 3 shows the percent of drug release over time.

TABLE 3
Percent of drug released over time

| | Number of Days | | | | | | | | | |
|---|---|---|---|---|---|---|---|---|---|---|
| | 1 | 2 | 3 | 4 | 7 | 8 | 9 | 15 | 22 | 29 |
| Sample 1 | 1.1% | 4.7% | 11.2% | 17.8% | 28.9% | 31.6% | 33.0% | 41.1% | 47.8% | 53.1% |
| Sample 2 | 0.5% | 1.5% | 3.9% | 6.5% | 12.9% | 14.7% | 16.0% | 22.5% | 29.1% | 34.0% |

As can be seen in FIG. 5 and Table 3, the drug or therapeutic agent released more quickly and to a greater extent when the polymer-drug composition was laminated between two layers of electrospun PTFE (Sample 1). In contrast, the small pore size ePTFE modulated the release of the drug in Sample 2.

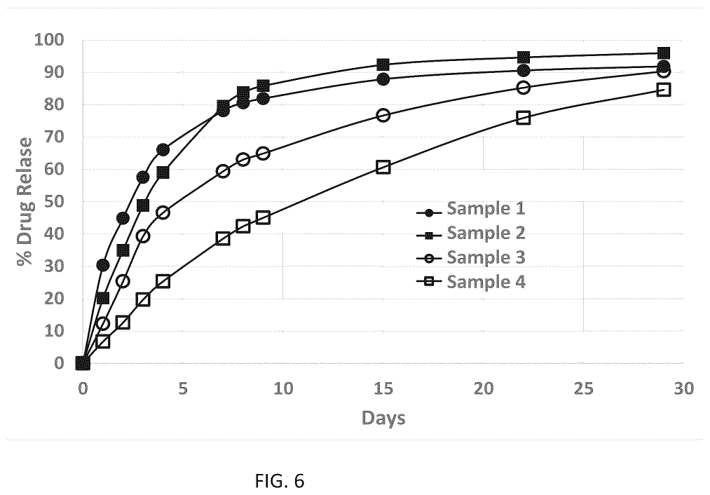

FIG. 6 is a graphical representation of experimental data providing relative drug release rates for samples made at least in part with a PET woven mesh and incubated in PBS (phosphate buffered saline) with 0.05% TWEEN® 20, pH 7.4 media (available from Sigma-Aldrich based in St. Louis, MO) at 37° C. Films of PVDF-HFP/rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 micrometers (μm) thick) was peeled off from the glass plate.

In a first sample (Sample 1), the polymer-drug composition was laminated between two layers of low-density woven mesh PET (pore size of approximately 180 μm). The same PET was used for each side of the lamination. In Sample 1, the therapeutic agent may release from both sides of the sample (two side release). In a second sample (Sample 2), the polymer-drug composition was laminated between two layers of high-density woven mesh PET (pore size in the range of about 10-20 μm). The same PET was used for each side of the lamination. In Sample 2, the therapeutic agent may release from both sides of the sample (two side release). A low-density woven mesh PET has larger pores than high-density woven mesh PET. In a third sample (Sample 3), the polymer-drug composition was laminated between a layer of low-density woven mesh PET and a layer of low density ePTFE. In Sample 3, the therapeutic agent may primarily release through the PET (one side release). In a fourth sample (Sample 4), the polymer-drug composition was laminated between a layer of high-density woven mesh PET and a layer of low density ePTFE. In Sample 4, the therapeutic agent may primarily release through the PET (one side release). Lamination of each sample was performed using a clamp force of approximately 1400 g at 200° C. for five minutes. Disks were cut from the laminate constructions and drug release was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 4 shows the percent of drug release over time.

TABLE 4
Percent of drug released over time

| | Number of Days | | | | | | | | | |
|---|---|---|---|---|---|---|---|---|---|---|
| | 1 | 2 | 3 | 4 | 7 | 8 | 9 | 15 | 22 | 29 |
| Sample | 30.3% | 44.8% | 57.5% | 66.0% | 78.1% | 80.5% | 81.8% | 87.8% | 90.5% | 91.8% |
| 1 | | | | | | | | | | |
| Sample | 20.1% | 34.9% | 48.8% | 59.0% | 79.5% | 83.7% | 85.8% | 92.3% | 94.6% | 95.9% |
| 2 | | | | | | | | | | |
| Sample | 12.2% | 25.3% | 39.3% | 46.4% | 59.4% | 63.0% | 64.9% | 76.6% | 85.2% | 90.3% |
| 3 | | | | | | | | | | |
| Sample | 6.7% | 12.6% | 19.7% | 25.3% | 38.5% | 42.3% | 45.0% | 60.6% | 75.9% | 84.6% |
| 4 | | | | | | | | | | |

As can be seen in Table 4 and FIG. 6, the large pore size of the woven mesh PET (both high-density and low-density) (Samples 1 and 2) and relatively large percentage of open area had less of a modulating effect on the drug release compared to ePTFE layers (Samples 3 and 4). Blocking drug release from one side by using a small pore size ePTFE reduced the drug release rate.

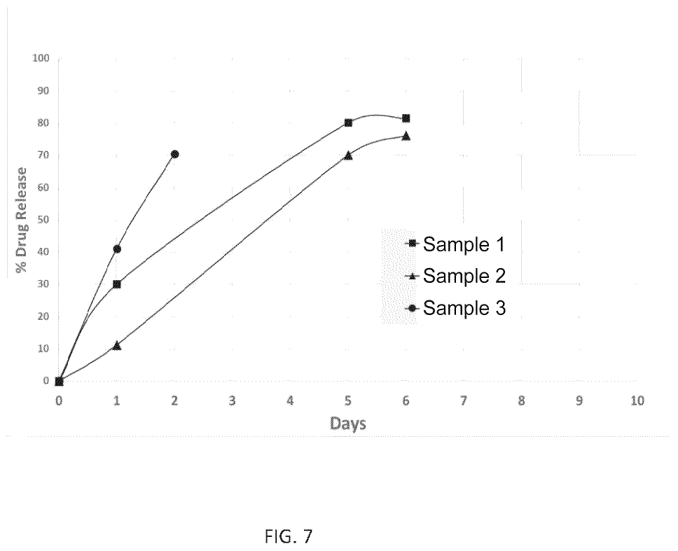

FIG. 7 is a graphical representation of experimental data providing relative drug release rates for samples made at least in part with microporous PVDF. Films of PVDF-HFP-rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 micrometers (μm) thick) was peeled off from the glass plate.

Microporous PVDF was prepared by coating a 10% solids solution of PVDF (500K MW, available from Sigma-Aldrich based in St. Louis, MO) onto a glass plate using a #20 Mayer coating bar. The coated plate was immediately immersed into a water bath at 40° C. for ten minutes. This resulted in non-solvent induced phase separation, leading to a microporous film. After removal from the water bath, the film was dried in a convection oven at 55° C.

In a first sample (Sample 1), the polymer-drug composition was laminated between two layers of microporous PVDF. The same microporous PVDF was used for each side of the lamination. In Sample 1, the therapeutic agent may release from both sides of the sample (two side release). In a second sample (Sample 2), the polymer-drug composition was laminated between a layer of microporous PVDF and a layer of low density ePTFE. In Sample 2, the therapeutic agent may primarily release through the PVDF (one side release). In a third sample (Sample 3), the polymer-drug composition was used as a control. Lamination of each sample was performed using a clamp force of approximately 1400 g at 200° C. for five minutes. Disks were cut from the laminate constructions and drug release was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 5 shows the percent of drug release over time.

TABLE 5
Percent of drug released over time

Number of Days

| | 1 | 2 | 5 | 6 |
|---|---|---|---|---|
| Sample 1 | 30.0% | — | 80.1% | 81.4% |
| Sample 2 | 11.2% | — | 70.1% | 76.1% |
| Sample 3 | 41.0% | 70.4% | — | — |
| (Control) | | | | |

As can be seen in Table 5 and FIG. 7, the microporous PVDF (Sample 1) allowed for the release of the therapeutic agent. Blocking drug release from one side by using a small pore size ePTFE (Sample 2) reduced the drug release rate.

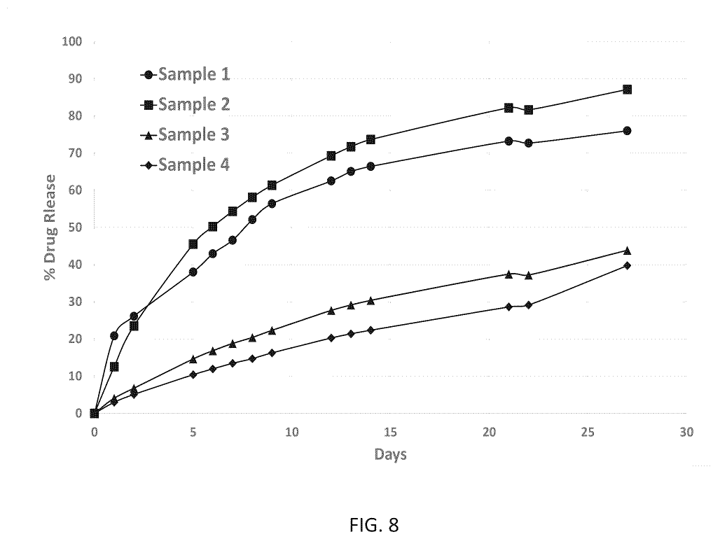

FIG. 8 is a graphical representation of experimental data providing relative drug release rates for samples made at least in part with ePTFE and a layer of PVDF-HFP. Films of PVDF-HFP-rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 micrometers (μm) thick) was peeled off from the glass plate.

The polymer-drug composition was laminated between a layer of small pore size ePTFE and a layer of PVDF-HFP. The thickness of the layer of PVDF-HFP for each sample was varied by cast coating PVDF-HFP from acetone at different percent solids and Mayer bar sizes. Table 6 shows the coating bar and layer thickness of the modulating layer of PVDF-HFP used for each sample.

TABLE 6
PVDF-HFP layer thickness

| Modulating | Coating | Calc coating |
|---|---|---|
| layer sample | Bar | thickness (μm) |
| 1 | #7 | 0.9 |
| 2 | #20 | 1.8 |
| 3 | #40 | 3.6 |
| 4 | #75 | 7.6 |

Lamination of each sample was performed using a clamp force of approximately 1400 g at 200° C. for five minutes. Disks were cut from the laminate constructions and drug release was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 7 shows the percent of drug release over time.

TABLE 7
Percent of drug released over time

| | Number of Days | | | | | | | | | | | |
|---|---|---|---|---|---|---|---|---|---|---|---|---|
| | 1 | 2 | 5 | 6 | 7 | 9 | 12 | 13 | 14 | 21 | 22 | 27 |
| Sample | 20.8% | 26.1% | 38.0% | 42.9% | 46.6% | 56.4% | 62.5% | 65.0% | 66.4% | 73.2% | 72.7% | 76.0% |
| 1 (#7 | | | | | | | | | | | | |
| bar) | | | | | | | | | | | | |
| Sample | 12.5% | 23.5% | 45.5% | 50.2% | 54.3% | 61.3% | 69.3% | 71.7% | 73.6% | 82.2% | 81.6% | 87.1% |
| 2 (#20 | | | | | | | | | | | | |
| bar) | | | | | | | | | | | | |
| Sample | 4.1% | 6.8% | 14.6% | 16.8% | 18.8% | 22.3% | 27.7% | 29.1% | 30.4% | 37.4% | 37.2% | 43.8% |
| 3 (#40 | | | | | | | | | | | | |
| bar) | | | | | | | | | | | | |
| Sample | 3.0% | 5.1% | 10.4% | 12.0% | 13.4% | 16.2% | 20.3% | 21.4% | 22.4% | 28.6% | 29.1% | 39.7% |
| 4 (#75 | | | | | | | | | | | | |
| bar) | | | | | | | | | | | | |

As can be seen in Table 7 and FIG. 8, increasing the thickness of the modulating PVDF-HFP generally decreased the drug release rate.

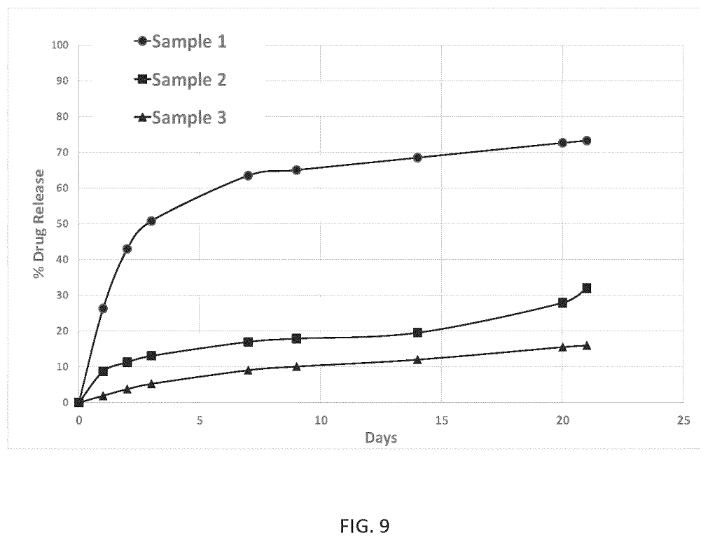

FIG. 9 is a graphical representation of experimental data providing relative drug release rates for samples made with ePTFE at varying lamination pressures. An unexpected finding in this invention is the impact of lamination pressure on drug release. The pressure used when laminating the ePTFE/PVDF-HFP-drug/ePTFE construct was found to have a significant impact on drug release. Films of PVDF-HFP-rivaroxaban (60/40 wt/wt) were prepared. For example, a ten-weight percent (wt %) solids solution of PVDF-HFP and rivaroxaban (60/40 wt/wt) in 60/40 acetone and DMF (wt/wt) solvent was coated on glass using a knife coater. The coating was dried at 125° C. for fifteen minutes in a convection oven. The resulting coating (approximately 14 μm thick) was peeled off from the glass plate.

The resulting polymer-drug compositions were laminated between two layers of ePTFE films each having a thickness of approximately 45 μm between two glass slides. The same ePTFE was used for both layers. The slides were clamped together with varying clamp forces ranging from 250 g force to 1400 g force and placed in an oven at 200° C. for five minutes. Disks were cut from the laminate constructions and drug release was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 8 shows the percent of drug release over time.

TABLE 8
Percent of drug released over time

| | Number of Days | | | | | | | |
|---|---|---|---|---|---|---|---|---|
| | 1 | 2 | 3 | 7 | 9 | 14 | 20 | 21 |
| Sample 1 | 26.3% | 42.9% | 50.7% | 63.4% | 65.0% | 68.4% | 72.6% | 73.2% |
| (1400 g | | | | | | | | |
| force) | | | | | | | | |
| Sample 2 | 8.7% | 11.3% | 13.1% | 17.0% | 17.9% | 19.5% | 27.9% | 32.0% |
| (1000 g | | | | | | | | |
| force) | | | | | | | | |
| Sample 3 | 1.9% | 3.8% | 5.3% | 9.0% | 10.1% | 12.0% | 15.5% | 16.0% |
| (250 g | | | | | | | | |
| force) | | | | | | | | |

As can be seen in Table 8 and FIG. 9, the drug release increased with increasing lamination pressure. This may be due to increased penetration of the drug layer into the pore structure of the ePTFE. Lamination pressure may similarly impact the drug release when polymers other than ePTFE are used.

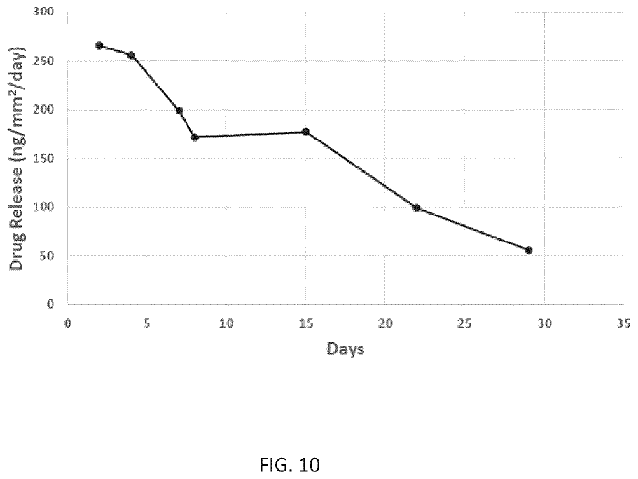

FIG. 10 is a graphical representation of in-vitro drug release rate (in nanograms (ng)/mm²/day) for a high-density PET/PVDF-HFP-rivaroxaban/ePTFE construction of this disclosure. The polymer-drug composition was laminated between a layer of high-density woven mesh PET and a layer of low density ePTFE. Based on previous work with a rivaroxaban eluting covered left atrial appendage device in a thrombogenic canine model, drug release down to approximately 10 ng/mm²/day was effective at preventing thrombus formation on the covered device. Table 9 shows the quantity of drug released over time.

TABLE 9
Quantity of drug released over time

| Day | Ng/mm²/day drug release |
|---|---|
| 2 | 265 |
| 4 | 256 |
| 7 | 199 |
| 8 | 172 |
| 15 | 177 |
| 22 | 99 |
| 29 | 56 |

The example of Table 9 and FIG. 10 shows greater than 10 ng/mm²/day at 30 days which is still in the effective release range for preventing thrombus formation on the surface of the device of the current disclosure.

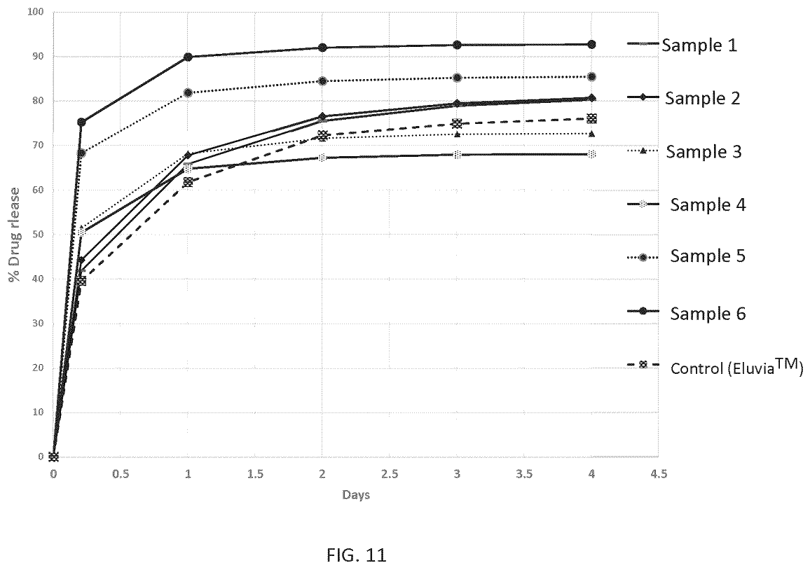

FIG. 11 is a graphical representation of experimental data providing relative drug release rates for a covered stent that is abluminally coated (e.g., radially outwardly coated) with a PVDF-HFP and paclitaxel drug coating composition. An exterior surface of an Innova™ nitinol stent (available from Boston Scientific based in Marlborough, MA) was coated with an outer covering of small pore size ePTFE such that the ePTFE spanned the open cells defined by the struts. Solutions of PVDF-HFP and paclitaxel were sprayed coated onto the radially outwards surface of ePTFE covered stents. A 3% solids solution of PVDF-HFP and paclitaxel (having a polymer to drug ratio of 90/10) dissolved in acetone was spray coated onto the ePTFE stent covering. Different samples were prepared with differing drug dose densities. The drug dose densities ranged from 0.18 μg paclitaxel per mm² to 0.48 μg paclitaxel per mm², as shown in Table 10A below.

TABLE 10A
Drug Dose Density

| Sample | Drug Dose Density |
|---|---|
| 1 | 0.41 μg/mm² |
| 2 | 0.48 μg/mm² |
| 3 | 0.37 μg/mm² |
| 4 | 0.34 μg/mm² |
| 5 | 0.21 μg/mm² |
| 6 | 0.18 μg/mm² |

Accelerated kinetic drug release (KDR) testing was done in sodium acetate buffer/isopropyl alcohol media (40/60 vol/vol) 45° C. Table 10B shows the percent of drug release over time.

TABLE 10B
Percent of drug released over time

| | Number of Days | | | | |
|---|---|---|---|---|---|
| | 0.21 | 1 | 2 | 3 | 4 |
| Sample 1 | 41.8% | 65.7% | 75.5% | 78.9 | 83.7 |
| Sample 2 | 44.2% | 67.7% | 76.5% | 79.4% | 83.6% |
| Sample 3 | 51.4% | 68.1% | 71.6% | 72.5% | 72.6% |
| Sample 4 | 50.4% | 64.8% | 67.2% | 67.9% | 68.0% |
| Sample 5 | 68.3% | 81.8% | 84.5% | 85.2% | 85.4% |
| Sample 6 | 75.2% | 89.9% | 92.0% | 92.6% | 92.7% |
| Control | 39.5% | 61.7% | 72.3% | 74.9% | 76.0% |

The release curves, as shown in FIG. 11, of the covered stent formulations are similar to the Eluvia™ paclitaxel eluting stent (control) (available from Boston Scientific based in Marlborough, MA).

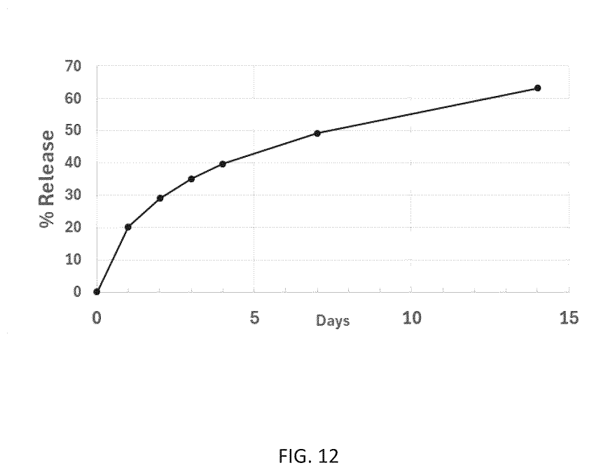

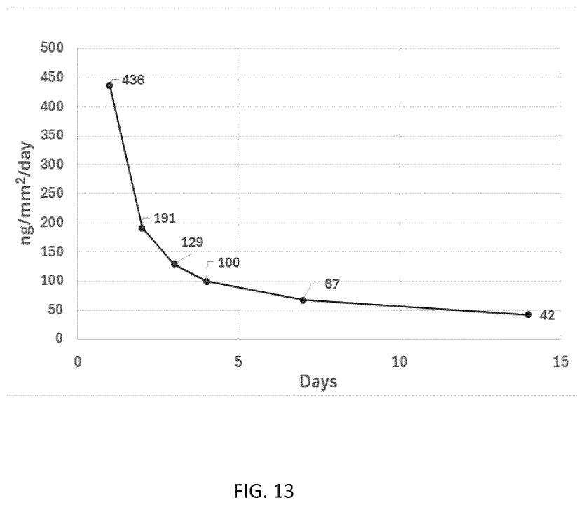

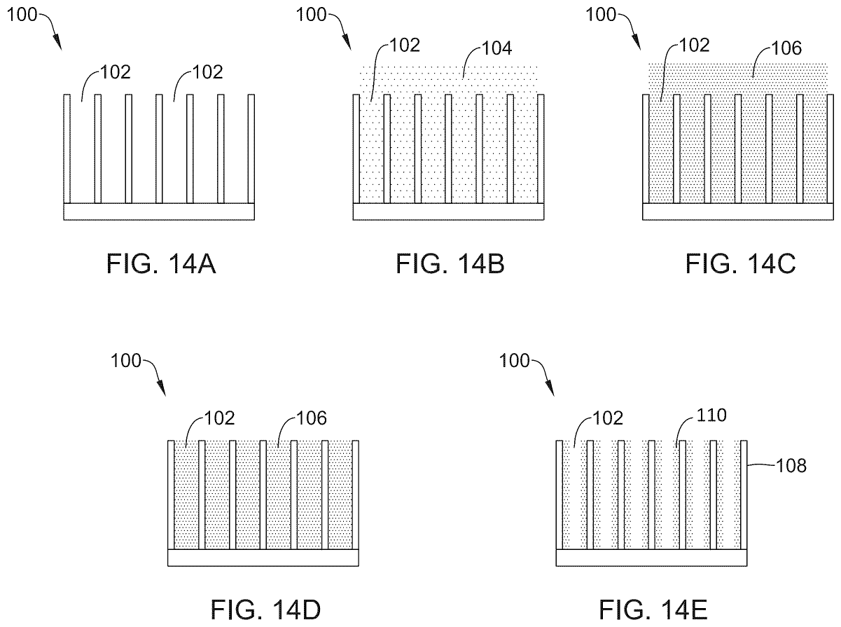

FIG. 12 is a graphical representation of experimental data providing in-vitro drug release rate (in percent of drug released) for an ePTFE covered stent (including both abluminal and luminal ePTFE coverings) in which the drug coating is contained within the pores of the ePTFE abluminal and luminal coverings. FIG. 13 is a graphical representation of experimental data providing in-vitro drug release rate (in nanograms (ng)/mm²/day) for the covered stent construction of the data of FIG. 12. FIGS. 14A-14E depict cross-sectional schematic views of the process steps for dip coating an ePTFE fabric to deposit a drug coating in the pores thereof. The same steps may be applied to an ePTFE covered stent to deposit a drug coating within the pores of the cPTFE. FIG. 14A depicts a schematic cross-sectional view of a portion of an ePTFE fabric 100 including a plurality of pores 102. For brevity and case of understanding not every pore is identified with a reference number. The ePTFE fabric 100 may be pre-wet with acetone 104. This may be achieved by dipping the ePTFE fabric into acetone such that acetone 104 flows into and is disposed within the pores 102 of the ePTFE fabric 100, as shown in FIG. 14B. Next, the pre-wet ePTFE fabric 100 may be dipped into a DMF/polymer/drug solution. In one illustrative example, the DMF/polymer/drug solution may be a DMF/PVDF-HFP/rivaroxaban solution. DMF and acetone are miscible allowing an exchange to equilibrium within the pores 102. The acetone 104 in the pores 102 of the ePTFE fabric 100 may be partially or fully displaced by the DMF/polymer/drug solution 106, as shown at FIG. 14C. For example, acetone 104 may diffuse out of the pores 102 as the DMF/polymer/drug solution 106 enters the pores 102. Next, the outer surface of the ePTFE fabric 100 may be squeegeed, or otherwise wiped, to remove excess surface coating, as shown in FIG. 14D. The ePTFE fabric 100 having the DMF/polymer/drug solution 106 disposed within the pores 102 thereof may then be dried to remove the solvent. After drying, the polymer/drug coating may not fill in an entirety of the void space of the pores 102, as shown in FIG. 14E. Rather, after drying, the polymer/drug coating 110 may coat the fabric fibrils 108 thus maintaining the open porous ePTFE structure.

Returning to FIGS. 12 and 13, stents including an abluminal and luminal ePTFE coating having a porosity of approximately 90% and internodal distance of about 20 μm were dip coated in a 15% solids solution of 80/20 PVDF-HFP/rivaroxaban (wt/wt) in dimethylformamide (DMF) after dipping the stents in acetone. DMF is required to solubilize rivaroxaban as DMF is the only organic solvent in which rivaroxaban has appreciable solubility. However, DMF does not wet out or wick into the ePTFE structure, thus preventing uptake of the polymer/drug into the pores of the ePTFE. However, it was found that pre-wetting the ePTFE with acetone (e.g., acetone wets out/wicks into the ePTFE porous structure), immediately followed by dipping the stent into the DMF/polymer/drug solution allows for the DMF/polymer/drug solution to equilibrate and migrate and/or penetrate into the pores of the ePTFE. For example, as the acetone diffuses out of the pores of the ePTFE, the DMF/polymer/drug solution enters the pores of the ePTFE. The resulting drug coating dose density was 2.2 μg drug/mm². To mitigate burst drug release, a topcoat of PVDF (8 wt % in ethyl acetate) was applied over the drug base coat by dip coating. The top coat coating density was 5.6 μg/mm². Excess surface coating from the inner diameter and the outer diameter of the stent was removed by squeegeeing both the inner diameter and the outer diameter of the stent immediately after dip coating both the drug layer and the topcoat layer (e.g., PVDF). Removal of excess surface coating was accomplished using a flexible silicone-based squeegee. Removing the excess surface coating (drug layer and/or topcoat layer) allows for preservation of the surface porosity after the coating is dried, which may be important for tissue integration and/or healing after stent implantation. The surface coating (drug layer and/or topcoat layer) may not fill in all of the void space of the ePFTE layers.

Drug release from the stents in which the drug coating is contained within the pores of the ePTFE abluminal and luminal coverings was measured by incubation in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography (HPLC). Table 11 shows the percent drug release over time and Table 12 shows the quantity of drug released over time.

TABLE 11
Percent of drug released over time

| | % Drug |
|---|---|
| Days | Release |
| 0 | 0 |
| 1 | 20.2 |
| 2 | 29.1 |
| 3 | 35.1 |
| 4 | 39.7 |
| 7 | 49.2 |
| 14 | 63.1 |

TABLE 12
Quantity of drug released over time

| | ng/mm²/day |
|---|---|
| Days | release |
| 1 | 436 |
| 2 | 191 |
| 3 | 129 |
| 4 | 100 |
| 7 | 67 |
| 14 | 42 |

As can be seen in FIGS. 12 and 13, as well as Tables 11 and 12, the average daily release of rivaroxaban is in the therapeutic range of greater than 10 ng/mm²/day up to and at fourteen days.

In another example, a 2% solids solution of PVDF-HFP and paclitaxel (having a polymer to drug ratio of 90/10) dissolved in acetone was spray coated onto an abluminal surface (outer surface) of four 6 mm diameter by 40 mm length (6 mm×40 mm) and four 7 mm diameter by 40 mm length (7 mm×40 mm) stents each including an ePTFE stent covering to form drug eluting covered stents. The paclitaxel dose density was 0.4 μg/mm². The stents included an ePTFE inner layer (e.g., similar to inner layer 18a of FIG. 2) and an ePTFE outer layer (e.g., similar to outer layer 18b of FIG. 2). The ePTFE covering extended from a proximal end to distal end of the stent to fully cover the stent. The drug eluting covered stents and control uncoated covered stents (having no therapeutic agent) were implanted in the superficial femoral artery (SFA) and the profunda artery of 25-45 kilogram (kg) pigs. The diameter of the SFA arteries were 3.5-4.5 mm. The diameter of the profunda arteries were 3.0-4.0 mm. The SFA and profunda treatment sites were first treated with a 20-mm long balloon inflated to give 40% vessel overstretch. This results in local vessel trauma over the balloon treatment site leading to stenosis. Immediately after balloon treatment, a covered stent was deployed so the distal end of the stent was centered over the balloon treatment site. The SFA arteries were implanted with 7 mm×40 mm drug eluting and 7 mm×40 mm control (no therapeutic agent) covered stents. The profunda arteries were treated with 6 mm×40 mm drug eluting and 6 mm×40 mm control covered stents. At 30 days, the percent restenosis was determined by angiography and intravascular ultrasound (IVUS) for the implanted stents. Table 13 shows the percent restenosis at 30 days.

TABLE 13
Percent Restenosis

| | Distal Edge | Proximal Edge |
|---|---|---|
| Test Article | Stenosis (%) | Stenosis (%) |
| Covered stent control (n = 4) | 39 | 61 |
| Drug eluting covered stent | 3 | 12 |
| (n = 4) | | |

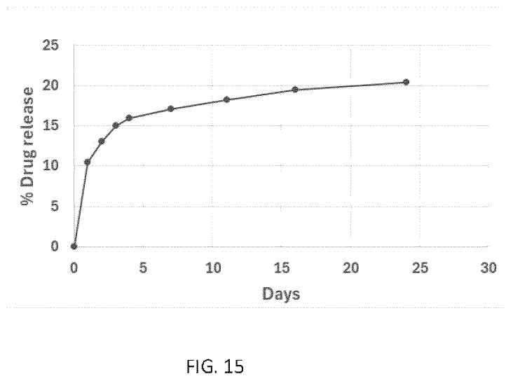

As shown in Table 13, the percent of restenosis of the drug eluting covered stents is significantly lower than the control stents (having no therapeutic agent) thus showing that the presence of abluminally coated paclitaxel is effective at reducing edge stenosis. FIG. 15 is a graphical representation of experimental data providing average drug release percentages for covered stents including an abluminal paclitaxel coating and Table 14 shows the in-vitro percent of drug released over time.

TABLE 14
Percent of drug released over time

| | % Drug |
|---|---|
| Days | Release |
| 0 | 0 |
| 1 | 10.5 |
| 2 | 13.1 |
| 3 | 15.0 |
| 4 | 15.9 |
| 7 | 17.0 |
| 11 | 18.2 |
| 16 | 19.5 |
| 24 | 20.4 |

In another example, fully covered stents including an abluminal and luminal paclitaxel coating were prepared. A 4% solids solution of PVDF-HFP and paclitaxel (having a polymer to drug ratio of 90/10) dissolved in acetone was dip coated onto an abluminal surface (outer surface) and luminal (inner surface) of eight stents (four 6 mm diameter by 40 mm length and four 7 mm diameter by 40 mm length each including an ePTFE stent covering to form drug eluting covered stents. The paclitaxel dose density was 0.2 μg/mm². The ePTFE covering extended from a proximal end to distal end of the stent to fully cover the stent. The stents included an ePTFE inner layer (e.g., similar to inner layer 18a of FIG. 2) and an ePTFE outer layer (e.g., similar to outer layer 18b of FIG. 2). The ePTFE fibril surface morphology was maintained after dip coating and drying.

Additionally, partially covered stents with drug coated ends were prepared. Eight partially ePTFE covered stents (four 6 mm diameter by 40 mm length and four 7 mm diameter by 40 mm length) in which the proximal and distal ends of the stent are uncovered or free from the ePTFE covering were spray coated with 2% solids solution of PVDF-HFP and paclitaxel (having a polymer to drug ratio of 90/10) dissolved in acetone. The uncovered portions of the proximal and distal ends each have a length of 4 mm. The partially covered stent was masked so that only the uncovered proximal and distal ends of the stent plus about 5 mm of the abluminal ePTFE covering adjacent each of the proximal and distal ends was coated with paclitaxel. Thus, about 9 mm of each end (proximal and distal) were coated with paclitaxel. The paclitaxel dose density was 0.4 μg/mm². The stents included an ePTFE inner layer (e.g., similar to inner layer 18a of FIG. 2) and an ePTFE outer layer (e.g., similar to outer layer 18b of FIG. 2). Each of the inner and outer layers extend less than an entire length of the stents.

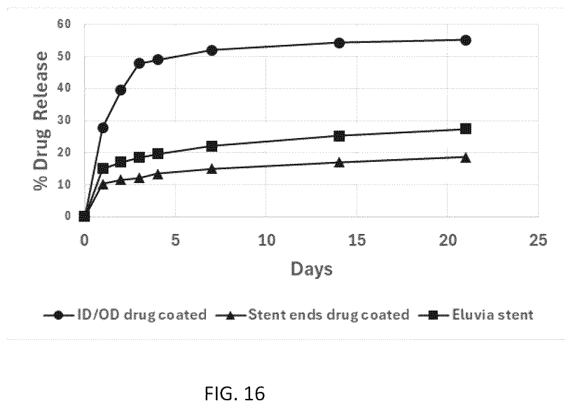

Drug release from the stents fully covered stents including an abluminal and luminal paclitaxel coating, the partially covered stents with drug coated ends, and an Eluvia™ control was measured by incubation in PBS (phosphate buffered saline) with 0.05% TWEEN® 20, pH 7.4 media (available from Sigma-Aldrich based in St. Louis, MO) at 37° C. in a shaker incubator. Drug content of the media at various time points was determined by high-performance liquid chromatography (HPLC). FIG. 16 is a graphical representation of experimental data providing average drug release percentages for the fully covered stents including an abluminal and luminal paclitaxel coating, the partially covered stents with drug coated ends, and an Eluvia™ control.

Additionally, drug eluting covered stents (fully and partially ePTFE covered), prepared as described with respect to FIG. 16, and uncoated fully and partially covered stents having no therapeutic agent and diameters of 6 mm and 7 mm and length of 40 mm were implanted in the superficial femoral artery and the profunda artery of pigs as described herein. At 30 days, the percent restenosis was determined by angiography and intravascular ultrasound (IVUS) for the implanted stents. Table 15 shows the percent restenosis. Both the fully covered stents including abluminal and luminal drug coatings and the stents with drug coated ends showed significantly less stenosis compared to the controls with no drug coatings. The stents including abluminal and luminal drug coatings have lower stenosis than the end coated stents which may be indicative of additional benefit to fully coating the luminal and abluminal surfaces of the covered stents with a therapeutic agent.

TABLE 15
Percent Restenosis

| | Distal | Proximal |
|---|---|---|
| | Edge | Edge |
| | Stenosis | Stenosis |
| Covered Stent | (%) | (%) |
| Fully ePTFE covered stent control (n = 4) | 64.0 | 60.2 |
| Fully ePTFE covered stent with ID and OD drug | 15.6 | 3.1 |
| coating (n = 4) | | |
| Partially ePTFE covered stent with uncovered | 60.2 | 50.0 |
| ends - control (n = 4) | | |
| Partially ePTFE covered stent with uncovered | 17.8 | 17.0 |
| ends - drug coated ends (n = 4) | | |

Table 16 shows the percent of drug released over time.

TABLE 16
Percent drug release over time

| | % Drug Release | | |
|---|---|---|---|
| | ID/OD drug coated | Covered stent with | Eluvia ™ comparative |
| Days | covered stent | ends drug coated | control |
| 0 | 0 | 0 | 0 |
| 1 | 27.9 | 10.2 | 15.0 |
| 2 | 39.4 | 11.5 | 17.0 |
| 3 | 47.8 | 12.0 | 18.5 |
| 4 | 49 | 13.3 | 19.6 |
| 7 | 52.0 | 15.0 | 22.0 |
| 14 | 54.4 | 17.0 | 25.2 |
| 21 | 55.2 | 18.6 | 27.4 |

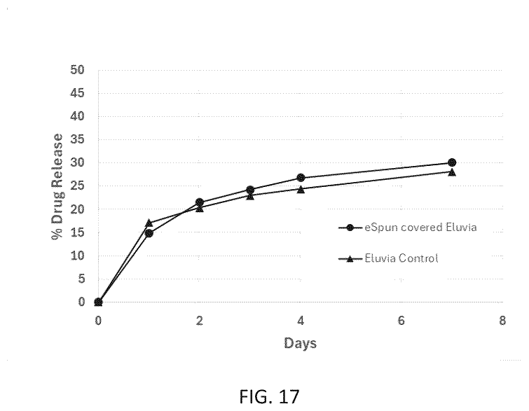

FIG. 17 is a graphical representation of in vitro drug release of an electrospun PTFE covered Eluvia™ stent compared to an uncovered Eluvia™ control. A commercial Boston Scientific paclitaxel eluting stent (Eluvia™) was covered with Bioweb™ electrospun PTFE (available from Zeus based in Orangeburg, South Carolina). The electrospun PTFE had an adhesive intermediate layer composed of electrospun polyurethane. The stent was encapsulated in an inner layer (luminal layer) and an outer layer (abluminal layer) of the electrospun PTFE by lamination at 200° C. The electrospun PTFE may provide flexibility to the covered stent. Table 17 shows the percent of drug released over time. As shown in FIG. 17 and Table 17, the electrospun PTFE covering does not significantly change the paclitaxel release profile of the Eluvia™ stent.

TABLE 17
Percent of drug released over time

% Drug Release

| | eSpun covered | Eluvia ™ comparative |
|---|---|---|
| Days | Eluvia ™ | control |
| 0 | 0 | 0 |
| 1 | 14.9 | 17.1 |
| 2 | 21.5 | 20.3 |
| 3 | 24.2 | 23.0 |
| 4 | 26.8 | 24.4 |
| 7 | 30.1 | 28.1 |

In another example, a solution of styrene-isobutylene-styrene block copolymer (SIBS) (15% solids in tetrahydrofuran (THF)) was coated on a Teflon™ coated PET film and dried at 125° C. to a dry thickness of about 20 μm. The SIBS layer is impermeable to cells and effectively impermeable to drug. A film of electrospun PTFE (Bioweb™ with the thermoplastic polyurethane (TPU) layer removed) was placed against the SIBS coating and laminated under pressure at 200° C. The laminated construct was then dip coated into a solution of acetone to wet out the electrospun PTFE and then immediately dip coated into a 15% solution of 80/20 PVDF-HFP/rivaroxaban in DMF. Excess polymer/drug coating was squeegeed off the surface of the electrospun PTFE and the coated construction was dried at 125° C. The resulting construct has the therapeutic agent (e.g., rivaroxaban) infused into the pores of the electrospun PTFE as described with respect to FIGS. 14A-14E. A PVDF-HFP topcoat was then applied to the construct by dip coating in 8% PVDF-HFP in ethyl acetate, squeegeeing off the excess coating, followed by drying at 125° C. The resulting coated composite was peeled off the Teflon/PET substrate giving the following structure: PVDF-HFP topcoat coated on PVDF-HFP/rivaroxaban with both the polymer topcoat and the polymer/drug composition coated within the electrospun PTFE layer, with the electrospun PTFE layer laminated to SIBS film.

In another example, a solution of styrene-isobutylene-styrene block copolymer (SIBS) (15% solids in tetrahydrofuran (THF) was coated on a second Teflon™ coated PET film and dried at 125° C. to a dry thickness of about 20 μm. A film of electrospun PTFE was placed against the SIBS coating and laminated under pressure at 200° C. to form a second laminated construct. The second laminated construct was dip coated into a 4% solids solution of 90/10 PVDF-HFP/paclitaxel in acetone followed by drying at 60° C. The resulting construct has the therapeutic agent (e.g., paclitaxel) infused into the pores of the electrospun PTFE. The resulting coated composite was peeled off the Teflon/PET substrate giving the following structure: PVDF-HFP/paclitaxel coated within the electrospun PTFE layer, with the electrospun PTFE layer laminated to SIBS film.

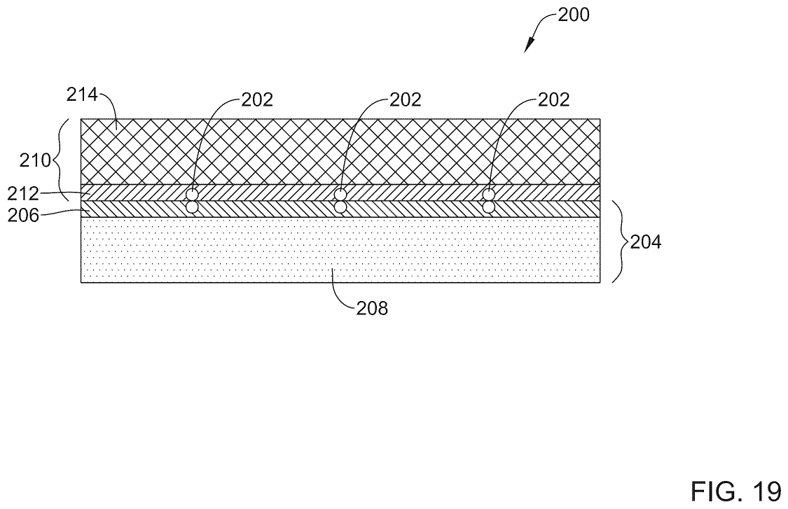

A covered stent was prepared by laminating at 200° C. a rivaroxaban infused electrospun PTFE/SIBS film on the inner of the stent and a paclitaxel infused electrospun PTFE/SIBS film on the outer diameter of the stent. The resulting covered stent releases rivaroxaban from the luminal surface of the stent to prevent stent thrombosis and the paclitaxel releases abluminally into the vessel tissue to prevent stenosis. A partial cross-sectional schematic of the covered stent 200 is shown in FIG. 19. As can be seen in FIG. 19, the resulting stent 200 includes an inner film 204 disposed along a luminal or inner surface of filaments or struts 202 forming the tubular body of the stent 200. The inner film 204 includes a first, non-therapeutic layer 206 and a second, therapeutic layer 208. In the example described above, the first non-therapeutic layer 206 is formed from SIBS. However, the first non-therapeutic layer 206 may be formed from other materials as desired. The material for the first non-therapeutic layer 206 may be selected to prevent flow or diffusion of cells or a therapeutic agent therethrough. In the example described above, the second therapeutic layer 208 is formed from drug infused electrospun PTFE. In the example, the second therapeutic layer 208 is infused with rivaroxaban. However, this is not required. The second therapeutic layer 208 may be infused with other drugs or therapeutic agents, as desired. The first non-therapeutic layer 206 is disposed radially outwards of the second therapeutic layer 208. This may allow the therapeutic agent to enter the bloodstream to reduce the risk of thrombus formation while preventing the therapeutic agent from being directed towards the vessel wall. While not explicitly shown, in some configurations, a polymeric topcoat may be disposed over an exposed surface of one or both the therapeutic layers 208, 214 to modulate a release of the therapeutic agent.

The resulting stent 200 further includes an outer film 210 disposed along an abluminal or outer surface of filaments or struts 202 forming the tubular body of the stent 200. The outer film 210 includes a first, non-therapeutic layer 212 and a second, therapeutic layer 214. In the example described above, the first non-therapeutic layer 212 is formed from SIBS. However, the first non-therapeutic layer 212 may be formed from other materials as desired. The material for the first non-therapeutic layer 212 may be selected to prevent flow or diffusion of cells or a therapeutic agent therethrough. In the example described above, the second therapeutic layer 214 is formed from drug infused electrospun PTFE. In the example, the second therapeutic layer 214 is infused with paclitaxel. However, the second therapeutic layer 214 may be infused with other drugs or therapeutic agents, as desired. The first non-therapeutic layer 212 is disposed radially inwards of the second therapeutic layer 214. This may allow the therapeutic agent to be directed towards the vessel wall to reduce the risk of stenosis while preventing the therapeutic agent from being directed towards the vessel lumen.

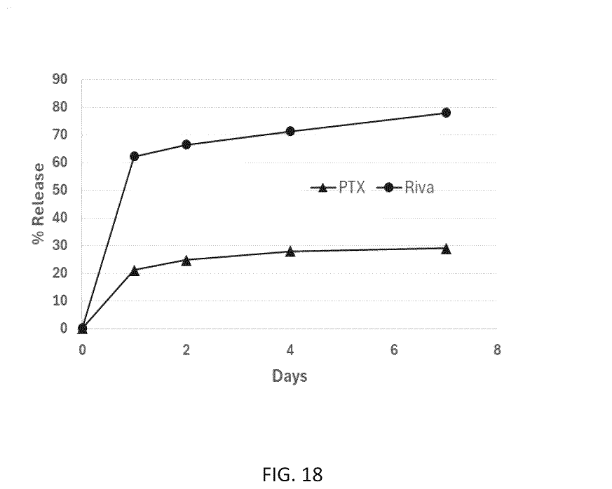

Drug release was measured by incubation of a covered stent including a therapeutic layer configured to release rivaroxaban from the luminal surface of the stent and a therapeutic layer configured to release paclitaxel abluminally in PBS (phosphate buffered saline) with 0.05% TWEEN® 20, pH 7.4 media (available from Sigma-Aldrich based in St. Louis, MO) at 37° C. Drug content of the media at various time points was determined by high-performance liquid chromatography. FIG. 18 and Table 18 shows the percent of drug release over time.

As can be seen in Table 18 and FIG. 18, both paclitaxel (PTX) and rivaroxaban were released from the respective therapeutic layers. The daily therapeutic dose of rivaroxaban is greater than 10 ng per mm² per day and the daily therapeutic dose of paclitaxel is greater than 0.3 ng per mm² per day, respectively. From Table 18 and FIG. 18, it can be seen that both rivaroxaban and paclitaxel are released above the therapeutic range. This example shows that rivaroxaban and paclitaxel can be independently formulated to release at therapeutic levels

TABLE 18
Percent of drug released over time

| | |
|---|---|
| % Drug Release | ng/mm²/day |

| Day | Paclitaxel | Rivaroxaban | Paclitaxel | Rivaroxaban |
|---|---|---|---|---|
| 1 | 21.1% | 62.2% | 91.4 | 588.2 |
| 2 | 24.7% | 66.4% | 16.3 | 40.1 |
| 4 | 27.9% | 71.3% | 6.8 | 23.1 |
| 7 | 28.9% | 78.0% | 3.4 | 21.0 |

The materials that can be used for the various components of the medical devices described herein may include those commonly associated with medical devices. The medical devices described herein may include components that may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super clastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear clastic and/or non-super-elastic nitinol may also be termed “substantially” linear elastic and/or non-super-clastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear clastic and/or non-super-clastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices described herein to achieve the same result.

In some embodiments, a degree of Magnetic Resonance Imaging (MRI) compatibility is imparted into the medical devices described herein. For example, the medical devices described herein, or portions thereof, may be made of a material that does not substantially distort the image and create substantial artifacts (i.e., gaps in the image). Certain ferromagnetic materials, for example, may not be suitable because they may create artifacts in an MRI image. The medical devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device, comprising:
an elongated tubular body having a strut framework defining a plurality of open cells;
a first polymeric film disposed on a luminal surface of the elongated tubular body, wherein the first polymeric film comprises electrospun polytetrafluoroethylene (PTFE); and
wherein a first therapeutic agent is infused within pores of the electrospun PTFE.

2. The medical device of claim 1, further comprising a second polymeric film disposed on an abluminal surface of the elongated tubular body, wherein the second polymeric film comprises a second therapeutic agent different from the first therapeutic agent.

3. The medical device of claim 1, wherein the first polymeric film comprises a drug-impermeable layer and a drug-containing layer disposed radially inward of the drug-impermeable layer.

4. The medical device of claim 2, wherein the second polymeric film comprises a drug-impermeable layer and a drug-containing layer disposed radially outward of the drug-impermeable layer.

5. The medical device of claim 3, wherein the drug-impermeable layer comprises styrene-isobutylene-styrene block copolymer (SIBS).

6. The medical device of claim 4, wherein the drug-impermeable layer comprises styrene-isobutylene-styrene block copolymer (SIBS).

7. The medical device of claim 2, wherein the first therapeutic agent comprises an anti-thrombotic agent and the second therapeutic agent comprises an anti-proliferative agent.

8. The medical device of claim 7, wherein the anti-thrombotic agent comprises rivaroxaban and the anti-proliferative agent comprises paclitaxel.

9. The medical device of claim 2, wherein the first polymeric film prevents diffusion of the first therapeutic agent toward an abluminal surface and the second polymeric film prevents diffusion of the second therapeutic agent toward a luminal surface.

10. The medical device of claim 2, wherein the second therapeutic agent is infused within pores of the second polymeric film.

11. The medical device of claim 2, further comprising a polymeric topcoat disposed over at least one of the first polymeric film or second polymeric film.

12. A method of manufacturing a drug-eluting medical device, comprising:
pre-wetting an expanded polytetrafluoroethylene (ePTFE) material with acetone;
immediately after pre-wetting, dip coating the ePTFE material in a solution comprising N,N-dimethyl formamide (DMF), a polymer, and a therapeutic agent;
removing excess coating from surfaces of the ePTFE material while preserving porosity; and
applying the coated ePTFE material to an elongated tubular body having a strut framework.

13. The method of claim 12, wherein the solution comprises poly(vinylidene fluoride)-co-hexafluoropropylene (PVDF-HFP) and rivaroxaban.

14. The method of claim 12, further comprising applying a topcoat over the coated ePTFE material to control a therapeutic agent release rate.

15. The method of claim 12, wherein removing excess coating comprises squeegeeing at least one of an inner or outer surface of the ePTFE material.

16. The method of claim 12, wherein the therapeutic agent equilibrates into pores of the ePTFE material as acetone diffuses out.

17. A stent, comprising:
an elongated tubular body having a strut framework defining a plurality of open cells;
a polymeric coating disposed on a surface of the elongated tubular body and extending across the plurality of open cells, the polymeric coating comprising a first inner layer disposed radially inward of the elongated tubular body and a second outer layer disposed radially outward of the elongated tubular body; and
a first drug coating composition disposed between the first inner layer and the second outer layer of the polymeric coating.

18. The stent of claim 17, wherein the first inner layer comprises a small pore size expanded polytetrafluoroethylene (ePTFE).

19. The stent of claim 17, wherein the second outer layer comprises a small pore size expanded polytetrafluoroethylene (ePTFE).

20. The stent of claim 17, further comprising a second drug coating composition disposed on an exterior surface of the second outer layer of the polymeric coating.
